(19) United States
(12) Patent Application Publication
(10) Pub. No.: US 2010/0297028 A1
(43) Pub. Date: Nov. 25, 2010

(54) MEDICAMENT COMBINATIONS FOR THE TREATMENT OF RESPIRATORY DISEASES

(75) Inventors: Ingo KONETZKI, Aachener-Oberforstbach (DE); Thierry BOUYSSOU, Warthausen (DE); Sabine PESTEL, Attenweiler (DE); Andreas SCHNAPP, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/848,364

(22) Filed: Aug. 2, 2010

Related U.S. Application Data
(63) Continuation of application No. 11/675,932, filed on Feb. 16, 2007, now abandoned.

(30) Foreign Application Priority Data
Feb. 16, 2006 (EP) 06110009.5

(51) Int. Cl. A61P 11/00; A61K 31/536; A61K 31/423; A61K 31/4709; A61K 31/56; A61P 11/06; A61P 11/08; A61P 31/12; A61P 31/04; A61K 9/72

(52) U.S. Cl. 424/40; 514/230.5; 514/375; 514/312; 514/171

(57) ABSTRACT

The present invention relates to new medicament combinations which contain in addition to one or more, preferably one compound of general formula 1

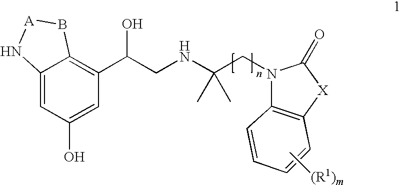

wherein A, B, R¹, X, n and m may have the meanings given in the claims and in the specification, at least one other active substance 2, processes for preparing them and their use as pharmaceutical compositions.

### SUMMARY OF THE INVENTION

The present invention relates to new medicament combinations, which contain at least one other active substance 2, in addition to one or more, preferably one compound of general formula 1

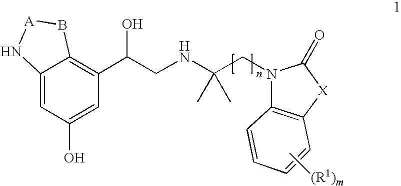

wherein A, B, R¹, X, n and m may have the meanings given in the claims and in the specification, to processes for preparing them and their use as pharmaceutical compositions.

### BRIEF DESCRIPTION OF THE DRAWING

FIG. 1: Exploded view of a preferred inhaler for administration of the pharmaceutical compositions described herein.

### DETAILED DESCRIPTION OF THE INVENTION

The present invention relates to medicament combinations, which, in addition to one or more, preferably one compound of general formula 1

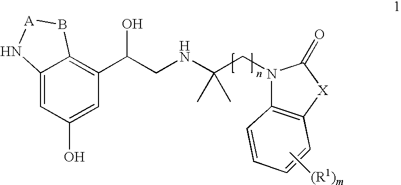

wherein
n denotes 1, 2, 3 or 4;
m denotes 1, 2 or 3;
X denotes CH₂, CO, NR², S or O;
A denotes a double-bonded group selected from among CO, SO or SO₂;
B denotes a double-bonded group selected from among O, S, CH₂, CR³R⁴—O, CR³R⁴—S, NR⁵, CR³R⁴—NR⁵, CH═CH or CH₂—CH₂;
R¹ denotes H, C₁₋₆-alkyl, C₂₋₆-alkenyl, C₂₋₆-alkynyl, C₃₋₆-cycloalkyl, C₁₋₆-haloalkyl, O—C₁₋₆-haloalkyl, halogen, OH, CN, NO₂, O—C₁₋₆-alkyl, COON or COO—C₁₋₄-alkyl;
R² denotes H, C₁₋₆-alkyl, C₁₋₄-alkylene-C₆-C₁₀-aryl or C₁₋₄-alkylene-C₃₋₆-cycloalkyl;
R³ denotes H or C₁₋₆-alkyl;
R⁴ denotes H or C₁₋₆-alkyl;
R⁵ denotes H or C₁₋₆-alkyl;

contain at least one other active substance 2.

Preferably the present invention relates to medicament combinations which contain as a further active substance 2 one or more compounds which are selected from among the categories of the anticholinergics (2a), PDEIV-inhibitors (2b), steroids (2c), LTD4-antagonists (2d) and EGFR inhibitors (2e), in addition to one or more, preferably one compound of formula 1.

Also preferred are the above medicament combinations, which contain at least one other active substance 2 in addition to one or more, preferably one compound of general formula 1, wherein A=CO.

Preferred are the above medicament combinations which contain, in addition to one or more, preferably one compound of general formula 1 wherein
n denotes 1, 2, 3 or 4;
m denotes 1, 2 or 3;
X denotes CH₂, CO, NR², S or O;
A denotes CO;
B denotes a double-bonded group selected from among O, S, CH₂, CR³R⁴—O, CR³R⁴—S, NR⁵, CR³R⁴—NR⁵, CH═CH or CH₂—CH₂;
R¹ denotes H, C₁₋₆-alkyl, C₁₋₆-haloalkyl, C₃₋₆-cycloalkyl, halogen, OH, CN, NO₂, O—C₁₋₆-alkyl, COON or COO—C₁₋₄-alkyl;
R² denotes H, C₁₋₄-alkyl, C₁₋₂-alkylene-C₃₋₆-cycloalkyl, phenylethyl or benzyl;
R³ denotes H or C₁₋₆-alkyl;
R⁴ denotes H or C₁₋₆-alkyl;
R⁵ denotes H or C₁₋₆-alkyl;

at least one other active substance 2.

Preferred are the above medicament combinations which contain, in addition to one or more, preferably one compound of general formula 1 wherein
n denotes 1, 2 or 3; preferably 2 or 3
m denotes 1, 2, 3 or 4; preferably 1.2 or 3;
X denotes CH₂, CO, NR², S or O;
A denotes CO;
B denotes a double-bonded group selected from among O, S, CH₂, CR³R⁴—O, CR³R⁴—S, NR⁵, CR³R⁴—NR⁵, CH═CH or CH₂—CH₂;
R¹ denotes H, C₁₋₄-alkyl, C₁₋₄-haloalkyl, cyclopropyl, cyclohexyl, halogen, OH, O—C₁₋₄-alkyl, COON or COOMe;
R² denotes H, C₁₋₄-alkyl, C₃₋₆-cycloalkyl-methyl, particularly preferably H, methyl or cyclopropylmethyl;
R³ denotes H or C₁₋₄-alkyl, preferably H or methyl;
R⁴ denotes H or C₁₋₄-alkyl, preferably H or methyl;
R⁵ denotes H or C₁₋₄-alkyl, preferably H or methyl;

at least one other active substance 2.

Also preferred are the above medicament combinations which contain in addition to one or more, preferably one compound of general formula 1 wherein
n denotes 2 or 3;
m denotes 1, 2 or 3;
X denotes CH₂, CO, NR², S or O;
A denotes CO;
B denotes a double-bonded group selected from among O, S, CH₂, CR³R⁴—O, CR³R⁴—S, NR⁵, CR³R⁴—NR⁵, CH═CH or CH₂—CH₂;
R¹ denotes H, methyl, ethyl, propyl, CF₃, CH₂F, CH₂CF₃, fluorine, chlorine, bromine, OH, methoxy, ethoxy, COON or COOMe;
R² denotes H, methyl, ethyl or propyl;
R³ denotes H, methyl, ethyl or propyl;
R⁴ denotes H, methyl, ethyl or propyl;
R⁵ denotes H, methyl, ethyl or propyl;

at least one other active substance 2.

Also preferred are the above medicament combinations which contain in addition to one or more, preferably one compound of general formula 1 wherein
n denotes 2 or 3;
m denotes 1, 2 or 3;
X denotes CH₂, CO, NR², S or O;
A denotes CO;
B denotes a double-bonded group selected from among O, S, CH₂, CR³R⁴—O, CR³R⁴—S, NR⁵, CR³R⁴—NR⁵, CH═CH or CH₂—CH₂;
R¹ denotes H, methyl, ethyl, propyl, CF₃, CH₂F, CH₂CF₃, fluorine, chlorine, bromine, OH, methoxy, ethoxy, COON or COOMe;
R² denotes H, methyl, ethyl or propyl;
R³ denotes H or methyl, preferably H;
R⁴ denotes H or methyl, preferably H;
R⁵ denotes H or methyl, preferably H;

at least one other active substance 2.

Also preferred are the above medicament combinations which contain in addition to one or more, preferably one compound of general formula 1 wherein
n denotes 2 or 3;
m denotes 1, 2 or 3;
X denotes CH₂, CO, NR², S or O;
A denotes CO;
B denotes a double-bonded group selected from among CH₂—O, CH═CH or CH₂—CH₂;
R¹ denotes H, methyl, ethyl, propyl, CF₃, CH₂F, CH₂CF₃, fluorine, chlorine, bromine, OH, methoxy, ethoxy, COON or COOMe;
R² denotes H, methyl, ethyl or propyl;

at least one other active substance 2.

Preferred according to the invention are the above medicament combinations which contain in addition to one or more, preferably one compound of general formula 1 wherein
n denotes 2 or 3;
m denotes 1 or 2;
X denotes CH₂, CO, NR², S or O;
A denotes CO;
B denotes a double-bonded group selected from among CH₂—O, CH═CH or CH₂—CH₂;
R¹ denotes H, methyl, ethyl, propyl, CF₃, CH₂F or CH₂CF₃;
R² denotes H, methyl, ethyl or propyl,

and R¹, R² and n may have the meanings given above,

at least one other active substance 2.

The present invention also relates to medicament combinations which contain in addition to one or more, preferably one compound of general formula 1, wherein
n denotes 2 or 3;
m denotes 1;
X denotes CH₂, CO, NR², S or O;
A denotes CO;
B denotes a double-bonded group selected from among CH₂—O, CH═CH or CH₂—CH₂;
R¹ denotes H, methyl or CF_{3;}
R² denotes H or methyl;

at least one other active substance 2.

The present invention also relates to medicament combinations which contain, in addition to one or more, preferably one compound of general formula 1 wherein
n denotes 2 or 3;
m denotes 1;
X denotes CH₂, CO, NR², S or O;
A denotes CO;
B denotes a double-bonded group selected from among CH₂—O, CH═CH or CH₂—CH₂;
R¹ denotes H, methyl or CF_{3;}
R² denotes H or methyl;

at least one other active substance 2.

In another preferred aspect the present invention relates to medicament combinations which contain, in addition to one or more, preferably one compound of general formula 1 wherein
X NR², O; wherein R² has the meaning given above;

at least one other active substance 2.

Particularly preferred are the above medicament combinations which contain, in addition to one or more, preferably one compound of general formula 1 wherein
n denotes 2 or 3;
m denotes 1;
X denotes NR², O;
A denotes CO;
B denotes a double-bonded group selected from among CH₂—O or CH═CH;
R¹ denotes H, methyl or CF_{3;}
R² denotes H or methyl;

at least one other active substance 2.

Particularly preferred are the above medicament combinations which contain in addition to one or more, preferably one compound of general formula 1, wherein
n denotes 2;
m denotes 1;
X denotes NH;
A denotes CO;
B denotes a double-bonded group CH₂—O;
R¹ denotes H, methyl or CF_{3;}

at least one other active substance 2.

Particularly preferred are the above medicament combinations which contain, in addition to one or more, preferably one compound of general formula 1 wherein
X denotes NR²;
R² denotes cyclopropylmethyl, cyclopropylethyl, cyclopentylmethyl, cyclopentylethyl, cyclohexylmethyl or cyclohexylethyl, preferably cyclopropylmethyl, cyclopentylmethyl or cyclohexylmethyl, particularly preferably cyclopropylmethyl

and wherein the groups n, m, A, B and R¹ may have the meanings given above,

at least one other active substance 2.

Particularly preferred are the above medicament combinations which contain, in addition to one or more, preferably one compound of general formula 1 wherein
X denotes NH

and wherein the groups n, m, A, B and R¹ may have the meanings given above,

at least one other active substance 2.

Particularly preferred are the above medicament combinations which contain, in addition to one or more, preferably one compound of general formula 1 wherein
X denotes CH₂

and wherein the groups n, m, A, B and R¹ may have the meanings given above,

at least one other active substance 2.

Particularly preferred are the above medicament combinations which contain, in addition to one or more, preferably one compound of general formula 1 wherein
X denotes CO

and wherein the groups n, m, A, B and R¹ may have the meanings given above,

at least one other active substance 2.

Particularly preferred are the above medicament combinations which contain, in addition to one or more, preferably one compound of general formula 1 wherein
X denotes O

and wherein the groups n, m, A, B and R¹ may have the meanings given above,

at least one other active substance 2.

Particularly preferred are the above medicament combinations which contain, in addition to one or more, preferably one compound of general formula 1 wherein
X denotes S

and wherein the groups n, m, A, B and R¹ may have the meanings given above,

at least one other active substance 2.

Compounds of formula 1, wherein A denotes CO and B denotes CH₂—O are characterised by general formula 1.1.

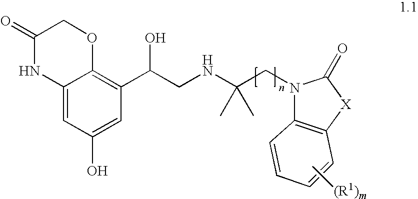

In a preferred aspect the present invention relates to medicament combinations which contain, in addition to one or more, preferably one compound of general formula 1.1 wherein n, m, X and R¹ may have the meanings given above,

at least one other active substance 2.

Compounds of formula 1 wherein A denotes CO and B denotes CH═CH are characterised by the general formula 1.2.

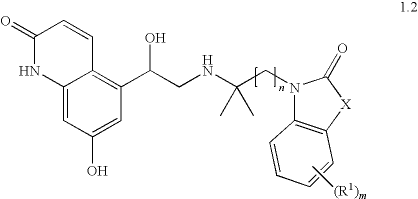

In a preferred aspect the present invention relates to medicament combinations which contain, in addition to one or more, preferably one compound of general formula 1.2 wherein n, m, X and R¹ may have the meanings given above,

at least one other active substance 2.

Compounds of formula 1, wherein A denotes CO and B denotes CH₂—CH₂, are characterised by the general formula 1.3.

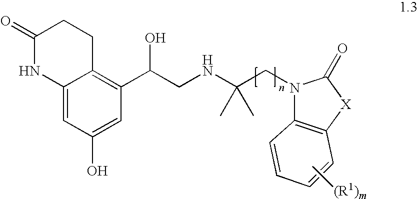

In a preferred aspect the present invention relates to medicament combinations which contain, in addition to one or more, preferably one compound of general formula 1.3

wherein n, m, X and R¹ may have the meanings given above,

at least one other active substance 2.

Compounds of formula 1, wherein A denotes CO and B denotes O, are characterised by the general formula 1.4.

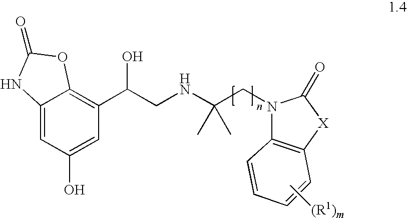

In a preferred aspect the present invention relates to medicament combinations which contain in addition to one or more, preferably one compound of general formula 1.4

wherein n, m, X and R¹ may have the meanings given above,

at least one other active substance 2.

Compounds of formula 1, wherein A denotes CO, B denotes CR³R⁴—O and R³ or R⁴ denotes methyl, are characterised by the general formula 1.5.

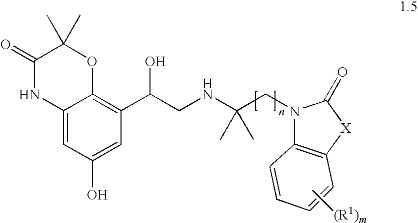

In a preferred aspect the present invention relates to medicament combinations which contain in addition to one or more, preferably one compound of general formula 1.5

wherein n, m, X and R¹ may have the meanings given above,

at least one other active substance 2.

In a preferred aspect the present invention relates to medicament combinations which contain at least one other active substance 2 in addition to one or more, preferably one compound of formula 1, which is selected from among

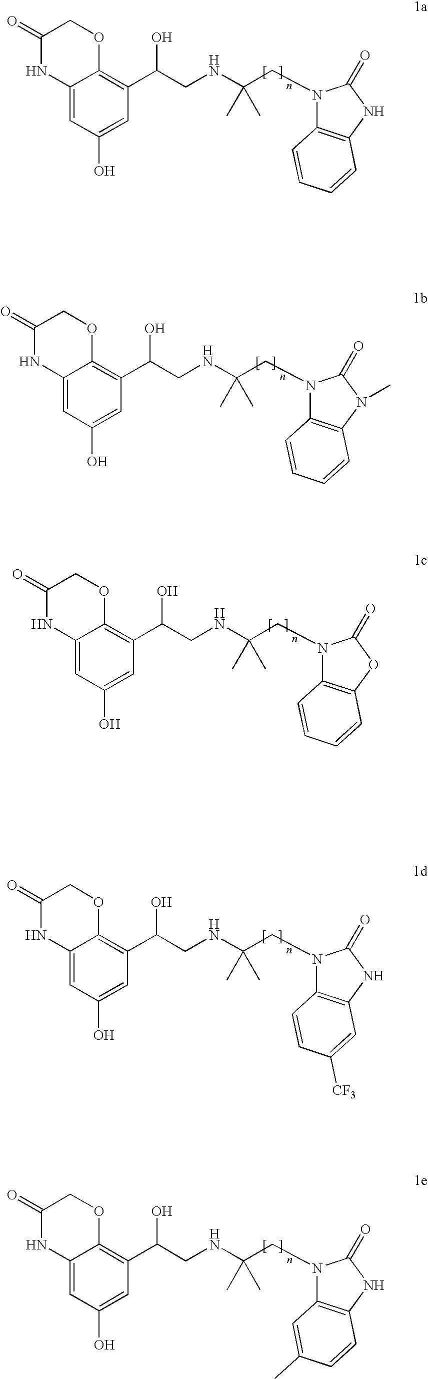

wherein for 1a, n=2 or 3 and for 1b, 1c, 1d and 1e, n=2 and the compounds optionally in the form of the individual enantiomers, mixtures of the individual enantiomers or racemates, optionally in the form of the acid addition salts thereof with pharmacologically acceptable acids as well as optionally in the form of the solvates and/or hydrates thereof.

In another aspect the present invention relates to the above-mentioned new compounds of formula 1 in the form of the individual optical isomers, mixtures of the individual enantiomers or racemates. Particularly preferably the compounds of formula 1 are in the form of the enantiomerically pure compounds, while the R-enantiomers of the compounds of formula 1 according to the invention are of exceptional importance. The R-enantiomers of the compounds of formula 1 may be represented by general formula R-1

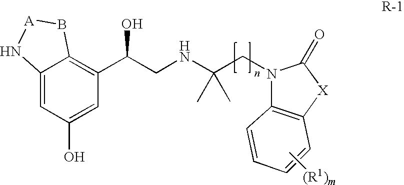

wherein the groups n, m, A, B, X and R¹ may have the meanings given above. Also particularly preferred among these are compounds of formula R-1 which are selected from among

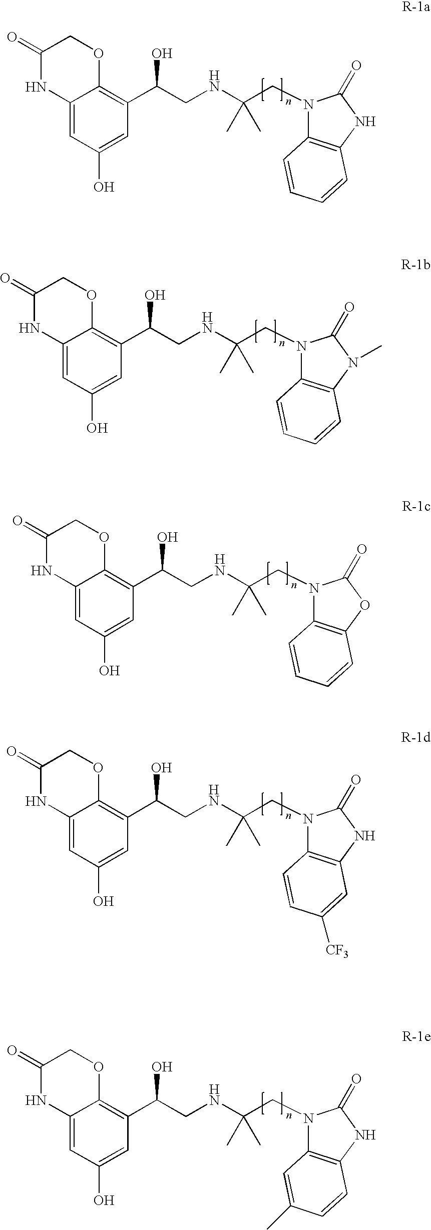

wherein in R-1a and R-1c, n=2 or 3, and in R-1b, R-1d and R-1e, n=2, and the compounds optionally in the form of the individual enantiomers, mixtures of the individual enantiomers or racemates, optionally in the form of the acid addition salts thereof with pharmacologically acceptable acids as well as optionally in the form of the solvates and/or hydrates thereof.

Methods of separating racemates into their respective enantiomers are known in the art and may be used analogously to prepare the enantiomerically pure R- or S-enantiomers of the compounds of formula 1.

Also particularly preferred are medicament combinations which contain at least one other active substance 2 in addition to one or more, preferably one compound of general formula 1 selected from the compounds

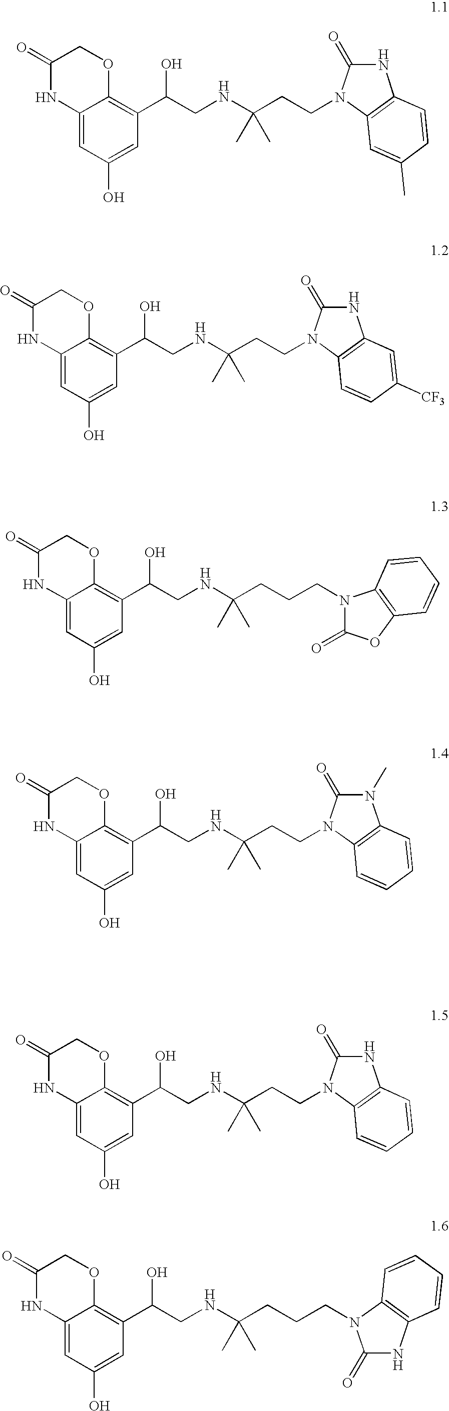

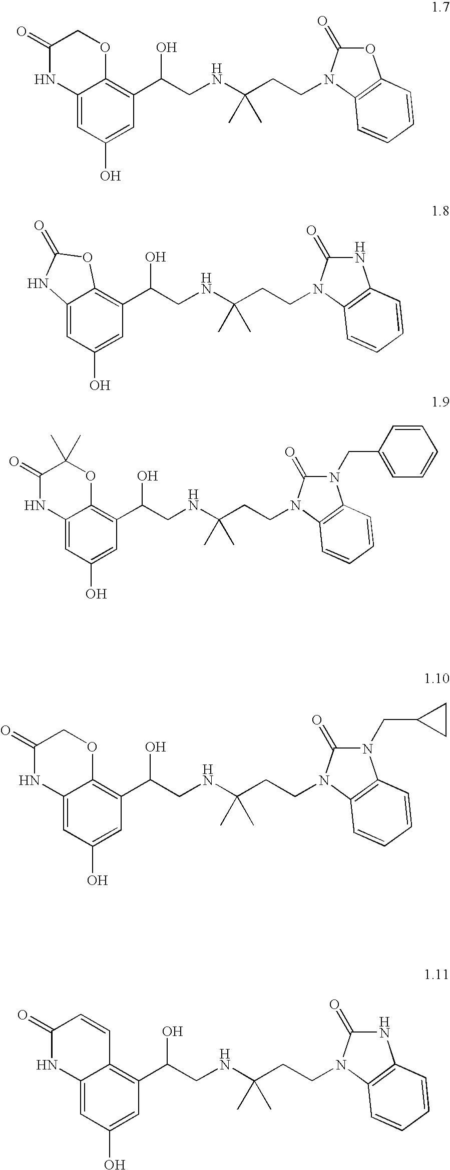

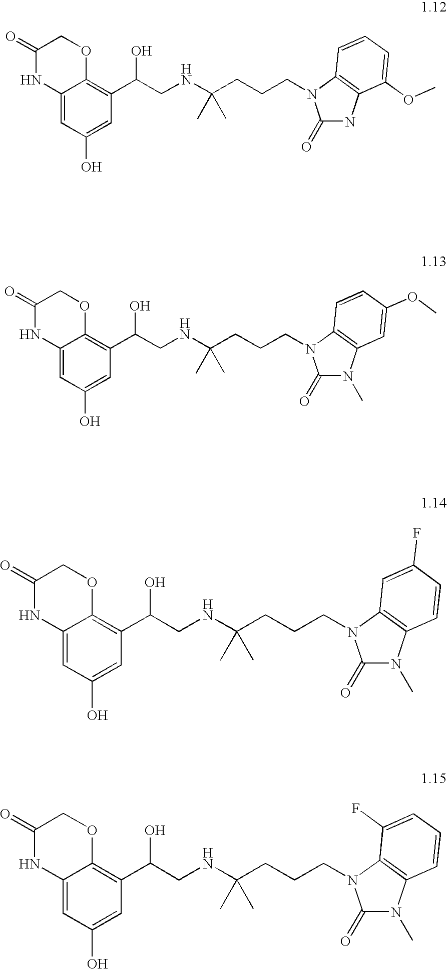

In another aspect the present invention relates to medicament combinations which contain the above-mentioned compounds of formula 1 in the form of the acid addition salts with pharmacologically acceptable acids as well as optionally in the form of the solvates and/or hydrates.

By acid addition salts with pharmacologically acceptable acids of the compounds 1 are meant for example salts selected from among the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate. Of the above-mentioned acid addition salts, the salts of hydrochloric acid, methanesulphonic acid, benzoic acid and acetic acid are particularly preferred according to the invention.

Preferred medicament combinations contain in addition to one or more, preferably one compound of formula 1 as a further active substance, one or more, preferably one anticholinergic 2a, optionally in combination with pharmaceutically acceptable excipients.

In the medicament combinations according to the invention, the anticholinergic 2a is preferably selected from among the tiotropium salts (2a.1), oxitropium salts (2a.2), flutropium salts (2a.3), ipratropium salts (2a.4), glycopyrronium salts (2a.5), trospium salts (2a.6) and the compounds of formulae 2a.7 to 2a.13.

In the above-mentioned salts 2a.1 to 2a.6 the cations tiotropium, oxitropium, flutropium, ipratropium, glycopyrronium and trospium are the pharmacologically active ingredients. Explicit reference to the above-mentioned cations is indicated by the terminology 2a.1′ to 2a.6′. Any reference to the above-mentioned salts 2a.1 to 2a.6 naturally also includes a reference to the corresponding cations tiotropium (2a.1′), oxitropium (2a.2′), flutropium (2a.3′), ipratropium (2a.4′), glycopyrronium (2a.5′), trospium (2a.6′).

By the salts 2a.1 to 2a.6 are meant according to the invention those compounds which contain in addition to the cations tiotropium (2a.1′), oxitropium (2a.2′), flutropium (2a.3′), ipratropium (2a.4′), glycopyrronium (2a.5′) and trospium (2a.6′) as counter-ion (anion) chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred. In the case of the trospium salts (2a.6) the chloride is particularly preferred. In the case of the other salts 2a.1 to 2a.5 the methanesulphonates and bromides are of particular importance. Of particular importance are medicament combinations which contain tiotropium salts (2a.1), oxitropium salts (2a.2) or ipratropium salts (2a.4), while the respective bromides are of particular importance according to the invention. The tiotropium bromide (2a.1) is of particular importance. The above-mentioned salts may optionally be present in the medicament combinations according to the invention in the form of the solvates or hydrates thereof, preferably in the form of their hydrates. In the case of tiotropium bromide the medicament combinations according to the invention preferably contain it in the form of the crystalline tiotropium bromide monohydrate which is known from WO 02/30928. If tiotropium bromide is used in anhydrous form in the medicament combinations according to the invention, preferably anhydrous crystalline tiotropium bromide is used, which is known from WO 03/000265.

Examples of novel preferred medicament combinations of preferred compounds of formula 1 with the above-mentioned anticholinergics 2a.1 to 2a.6 are combinations containing the compounds 1.1 and 2a.1; 1.1 and 2a.2; 1.1 and 2a.3; 1.1 and 2a.4; 1.1 and 2a.5; 1.1 and 2a.6; 1.2 and 2a.1; 1.2 and 2a.2; 1.2 and 2a.3; 1.2 and 2a.4; 1.2 and 2a.5; 1.2 and 2a.6; 1.3 and 2a.1; 1.3 and 2a.2; 1.3 and 2a.3; 1.3 and 2a.4; 1.3 and 2a.5; 1.3 and 2a.6; 1.4 and 2a.1; 1.4 and 2a.2; 1.4 and 2a.3; 1.4 and 2a.4; 1.4 and 2a.5; 1.4 and 2a.6; 1.5 and 2a.1; 1.5 and 2a.2; 1.5 and 2a.3; 1.5 and 2a.4; 1.5 and 2a.5; 1.5 and 2a.6; 1.6 and 2a.1; 1.6 and 2a.2; 1.6 and 2a.3; 1.6 and 2a.4; 1.6 and 2a.5; 1.6 and 2a.6; 1.7 and 2a.1; 1.7 and 2a.2; 1.7 and 2a.3; 1.7 and 2a.4; 1.7 and 2a.5; 1.7 and 2a.6; 1.12 and 2a.1; 1.12 and 2a.2; 1.12 and 2a.3; 1.12 and 2a.4; 1.12 and 2a.5; 1.12 and 2a.6; 1.14 and 2a.1; 1.14 and 2a.2; 1.14 and 2a.3; 1.14 and 2a.4; 1.14 and 2a.5; 1.14 and 2a.6; 1.15 and 2a.1; 1.15 and 2a.2; 1.15 and 2a.3; 1.15 and 2a.4; 1.15 and 2a.5 or 1.15 and 2a.6 in each case optionally in the form of the racemates, enantiomers or diastereomers thereof and optionally in the form of the pharmacologically acceptable acid addition salts, solvates and/or hydrates thereof.

Of the combinations mentioned above, the preferred ones according to the invention are those which contain one of the compounds 1.2, 1.5, 1.8, 1.10, 1.12 or 1.15 as the compound of formula 1. Also preferred, of the combinations mentioned above, according to the invention, are those which contain one of the compounds 2a.1, 2a.2 or 2a.4 as the compound 2a, while those combinations which contain the compound 2a.1 are particularly important according to the invention.

Optionally the above-mentioned anticholinergics have chiral carbon centres. In this case the medicament combinations according to the invention may contain the anticholinergics in the form of their enantiomers, mixtures of enantiomers or racemates, while enantiomerically pure anticholinergics are preferably used.

In another preferred embodiment of the present invention the anticholinergics 2a contained in the medicament combinations according to the invention are selected from the salts of formula 2a.7

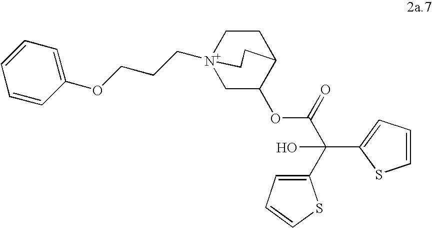

wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,

optionally in the form of the racemates, enantiomers or hydrates thereof. Preferred medicament combinations contain salts of formula 2a.7, wherein
X⁻ denotes an anion with a single negative charge, preferably an anion selected from among the fluoride, chloride, bromide, methanesulphonate and p-toluenesulphonate, preferably bromide,

optionally in the form of the racemates, enantiomers or hydrates thereof. Preferred medicament combinations contain salts of formula 2a.7, wherein
X⁻ denotes an anion with a single negative charge, preferably an anion selected from among the chloride, bromide and methanesulphonate, preferably bromide,

optionally in the form of the racemates, enantiomers or hydrates thereof. Particularly preferred medicament combinations contain the compound of formula 2a.7 in the form of the bromides. Of particular importance are those medicament combinations which contain the enantiomers of formula 2a.7-ene

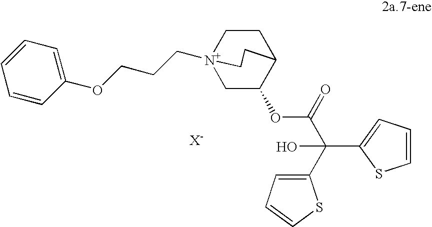

wherein X⁻ may have the meanings given above.

Examples of novel medicament combinations of preferred compounds of formula 1 with the above-mentioned anticholinergics 2a.7 are combinations containing the compounds 1.2 and 2a.7; 1.2 and 2a.7-ene; 1.5 and 2a.7; 1.5 and 2a.7-ene; 1.8 and 2a.7; 1.8 and 2a.7-ene; 1.10 and 2a.7; 1.10 and 2a.7-ene; 1.12 and 2a.7; 1.12 and 2a.7-ene; 1.15 and 2a.7 or 1.15 and 2a.7-ene; in each case optionally in the form of the racemates, enantiomers or diastereomers thereof and optionally in the form of the pharmacologically acceptable acid addition salts, solvates and/or hydrates thereof.

In another preferred embodiment of the present invention the anticholinergics 2a contained in the medicament combinations according to the invention are selected from the salts of formula 2a.8

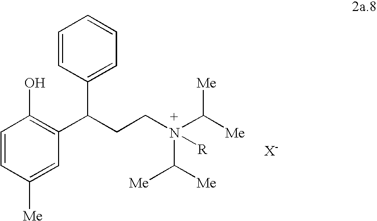

wherein R denotes either methyl (2a.8.1) or ethyl (2a.8.2) and wherein X⁻ may have the meanings given above. In an alternative embodiment the compound of formula 2a.8 is present in the form of the free base 2a.8-base

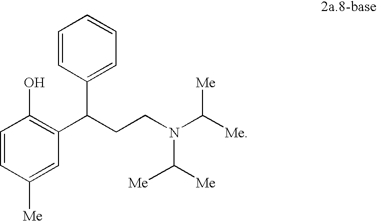

The medicament combinations according to the invention may contain the anticholinergic of formula 2a.8 (or 2a.8-base) in the form of the enantiomers, mixtures of enantiomers or racemates thereof. Preferably the anticholinergics of formula 2a.8 (or 2a.8-base) are present in the form of their R-enantiomers.

Examples of novel medicament combinations of preferred compounds of formula 1 with the above-mentioned anticholinergics 2a.8 are combinations containing the compounds 1.2 and 2a.8.1; 1.2 and 2a.8.2; 1.5 and 2a.8.1; 1.5 and 2a.8.2; 1.8 and 2a.8.1; 1.8 and 2a.8.2; 1.10 and 2a.8.1; 1.10 and 2a.8.2; 1.12 and 2a.8.1; 1.12 and 2a.8.2; 1.15 and 2a.8.1 or 1.15 and 2a.8.2, in each case optionally in the form of the racemates, enantiomers or diastereomers thereof and optionally in the form of the pharmacologically acceptable acid addition salts, solvates and/or hydrates thereof.

In another preferred embodiment of the present invention the anticholinergics 2a contained in the medicament combinations according to the invention are selected from the compounds of formula 2a.9

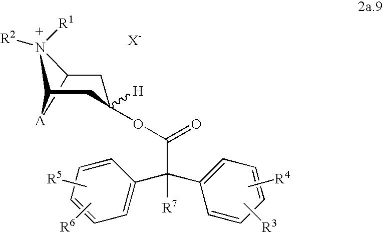

wherein
A denotes a double-bonded group selected from the groups

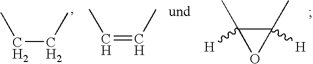

X⁻ denotes one of the above-mentioned anions with a single negative charge, preferably chloride, bromide or methanesulphonate,
R¹ and R² which may be identical or different denotes a group selected from methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂F, —O—CH₂CH₂F, —CH₂OH, —CH₂CH₂OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine.

The compounds of formula 2a.9 are known in the art (WO 02/32899).

Within the scope of the medicament combinations according to the invention preferred compounds of formula 2a.9 are those wherein
X⁻ denotes bromide;
R¹ and R² which may be identical or different denote methyl or ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine.

Of particular importance are medicament combinations which contain those compounds of formula 2a.9, wherein
A denotes a double-bonded group selected from

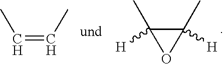

Of particular importance are those medicament combinations which contain in addition to a compound of formula 1 one of the following compounds of formula 2a.9:
tropenol 2,2-diphenylpropionate-methobromide (2a.9.1),
scopine 2,2-diphenylpropionate-methobromide (2a.9.2),
scopine 2-fluoro-2,2-diphenylacetate-methobromide (2a.9.3),
tropenol 2-fluoro-2,2-diphenylacetate-methobromide (2a.9.4);

The compounds of formula 2a.9 may optionally be present in the form of their enantiomers, mixtures of their enantiomers or racemates, as well as optionally in the form of the hydrates and/or solvates thereof.

Examples of novel medicament combinations of preferred compounds of formula 1 with the above-mentioned anticholinergics 2a.9 are combinations containing the compounds 1.1 and 2a.9.1; 1.1 and 2a.9.2; 1.1 and 2a.9.3; 1.1 and 2a.9.4; 1.2 and 2a.9.1; 1.2 and 2a.9.2; 1.2 and 2a.9.3; 1.2 and 2a.9.4; 1.3 and 2a.9.1; 1.3 and 2a.9.2; 1.3 and 2a.9.3; 1.3 and 2a.9.4; 1.4 and 2a.9.1; 1.4 and 2a.9.2; 1.4 and 2a.9.3; 1.4 and 2a.9.4; 1.5 and 2a.9.1; 1.5 and 2a.9.2; 1.5 and 2a.9.3; 1.5 and 2a.9.4; 1.6 and 2a.9.1; 1.6 and 2a.9.2; 1.6 and 2a.9.3; 1.6 and 2a.9.4; 1.7 and 2a.9.1; 1.7 and 2a.9.2; 1.7 and 2a.9.3; 1.7 and 2a.9.4; 1.12 and 2a.9.1; 1.12 and 2a.9.2; 1.12 and 2a.9.3; 1.12 and 2a.9.4; 1.14 and 2a.9.1; 1.14 and 2a.9.2; 1.14 and 2a.9.3; 1.14 and 2a.9.4; 1.15 and 2a.9.1; 1.15 and 2a.9.2; 1.15 and 2a.9.3; 1.15 and 2a.9.4, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.15. Also preferred, of the above-mentioned combinations according to the invention, are those which contain as compound 2a.9 one of the compounds 2a.9.1 or 2a.9.2, while those combinations which contain the compound 2a.9.2, are particularly important according to the invention.

In another preferred embodiment of the present invention the anticholinergics 2a contained in the medicament combinations according to the invention are selected from the compounds of formula 2a.10

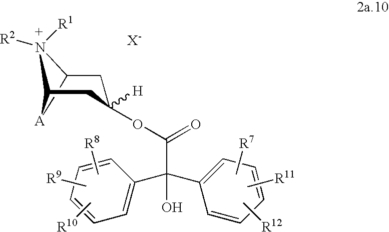

wherein
A, X, R¹ and R² may have the meanings given above and wherein
R⁷, R⁸, R⁹, R¹⁰, R¹¹ and R¹², which may be identical or different, represent hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen.

The compounds of formula 2a.10 are known in the art (WO 02/32898).

Within the scope of the medicament combinations according to the invention preferred compounds of formula 2a.10 are those wherein
A denotes a double-bonded group selected from

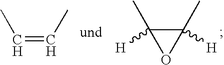

X⁻ denotes bromide;
R¹ and R² which may be identical or different, denote methyl or ethyl, preferably methyl;
R⁷, R⁸, R⁹, R¹⁰, R¹¹ and R¹², which may be identical or different, denote hydrogen, fluorine, chlorine or bromine, preferably fluorine, while at least one of the groups R⁷, R⁸, R⁹, R¹⁰, R¹¹ and R¹² may not be hydrogen.

Of particular importance are those medicament combinations which contain, in addition to a compound of formula 1, one of the following compounds of formula 2a.10:
tropenol 3,3′,4,4′-tetrafluorobenzilate-methobromide (2a.10.1),
scopine 3,3′,4,4′-tetrafluorobenzilate-methobromide (2a.10.2),
tropenol 4,4′-difluorobenzilate-methobromide (2a.10.3),
scopine 4,4′-difluorobenzilate-methobromide (2a.10.4),
tropenol 3,3′-difluorobenzilate-methobromide (2a.10.5),
scopine 3,3′-difluorobenzilate-methobromide (2a.10.6).

The compounds of formula 2a.10 may optionally be present in the form of the enantiomers, mixtures of enantiomers or racemates thereof, as well as optionally in the form of the hydrates and/or solvates thereof.

Examples of novel medicament combinations of preferred compounds of formula 1 with the above-mentioned anticholinergics 2a.10 are combinations containing the compounds 1.1 and 2a.10.1; 1.1 and 2a.10.2; 1.1 and 2a.10.3; 1.1 and 2a.10.4; 1.1 and 2a.10.5; 1.1 and 2a.10.6; 1.2 and 2a.10.1; 1.2 and 2a.10.2; 1.2 and 2a.10.3; 1.2 and 2a.10.4; 1.2 and 2a.10.5; 1.2 and 2a.10.6; 1.3 and 2a.10.1; 1.3 and 2a.10.2; 1.3 and 2a.10.3; 1.3 and 2a.10.4; 1.3 and 2a.10.5; 1.3 and 2a.10.6; 1.4 and 2a.10.1; 1.4 and 2a.10.2; 1.4 and 2a.10.3; 1.4 and 2a.10.4; 1.4 and 2a.10.5; 1.4 and 2a.10.6; 1.5 and 2a.10.1; 1.5 and 2a.10.2; 1.5 and 2a.10.3; 1.5 and 2a.10.4; 1.5 and 2a.10.5; 1.5 and 2a.10.6; 1.6 and 2a.10.1; 1.6 and 2a.10.2; 1.6 and 2a.10.3; 1.6 and 2a.10.4; 1.6 and 2a.10.5; 1.6 and 2a.10.6; 1.7 and 2a.10.1; 1.7 and 2a.10.2; 1.7 and 2a.10.3; 1.7 and 2a.10.4; 1.7 and 2a.10.5; 1.7 and 2a.10.6; 1.12 and 2a.10.1; 1.12 and 2a.10.2; 1.12 and 2a.10.3; 1.12 and 2a.10.4; 1.12 and 2a.10.5; 1.12 and 2a.10.6; 1.14 and 2a.10.1; 1.14 and 2a.10.2; 1.14 and 2a.10.3; 1.14 and 2a.10.4; 1.14 and 2a.10.5; 1.14 and 2a.10.6; 1.15 and 2a.10.1; 1.15 and 2a.10.2; 1.15 and 2a.10.3; 1.15 and 2a.10.4; 1.15 and 2a.10.5; 1.15 and 2a.10.6, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.15. Also preferred, of the above-mentioned combinations according to the invention, are those which contain as compound 2a.10 one of the compounds 2a.10.1, 2a.10.2, 2a.10.3 or 2a.10.4, while those combinations which contain the compounds 2a.10.1 or 2a.10.2 are particularly important according to the invention.

In another preferred embodiment of the present invention the anticholinergics 2a contained in the medicament combinations according to the invention are selected from the compounds of formula 2a.11

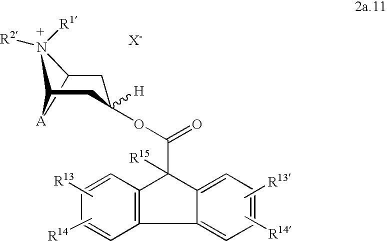

wherein
A and X⁻ may have the meanings given above and wherein
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen, or
   R^{1′} and R^{2′} together denote a —C₃-C₅-alkylene bridge;
R¹³, R¹⁴, R^{13′} and R^{14′} which may be identical or different, represent hydrogen, —C₁-C₄-alkyl, —C₁-C₄-alkyloxy, hydroxy, —CF₃, —CHF₂, CN, NO₂ or halogen.

The compounds of formula 2a.11 are known in the art (WO 03/064419).

Within the scope of the medicament combinations according to the invention preferred compounds of formula 2a.11 are those wherein
A denotes a double-bonded group selected from

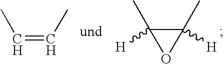

X⁻ denotes an anion selected from chloride, bromide and methanesulphonate, preferably bromide;
R¹⁵ denotes hydroxy, methyl or fluorine, preferably methyl or hydroxy;
R^{1′} and R^{2′} which may be identical or different denote methyl or ethyl, preferably methyl;
R¹³, R¹⁴, R^{13′} and R^{14′} which may be identical or different denote hydrogen, —CF₃, —CHF₂ or fluorine, preferably hydrogen or fluorine.

Within the scope of the medicament combinations according to the invention particularly preferred compounds of formula 2a.11 are those wherein
A denotes a double-bonded group selected from

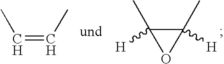

X⁻ denotes bromide;
R¹⁵ denotes hydroxy or methyl, preferably methyl;
R^{1′} and R^{2′} which may be identical or different denote methyl or ethyl, preferably methyl;
R¹³, R¹⁴, R^{13′} and R^{14′} which may be identical or different denote hydrogen or fluorine.

Of particular importance are those medicament combinations which contain, in addition to a compound of formula 1, one of the following compounds of formula 2a.11:
tropenol 9-hydroxy-fluorene-9-carboxylate methobromide (2a.11.1);
tropenol 9-fluoro-fluorene-9-carboxylate methobromide (2a.11.2);
scopine 9-hydroxy-fluorene-9-carboxylate methobromide (2a.11.3);
scopine 9-fluoro-fluorene-9-carboxylate methobromide (2a.11.4);
tropenol 9-methyl-fluorene-9-carboxylate methobromide (2a.11.5);
scopine 9-methyl-fluorene-9-carboxylate methobromide (2a.11.6);

The compounds of formula 2a.11 may optionally be present in the form of the enantiomers, mixtures of enantiomers or racemates thereof, as well as optionally in the form of the hydrates and/or solvates thereof.

Examples of novel medicament combinations of preferred compounds of formula 1 with the above-mentioned anticholinergics 2a.11 are combinations containing the compounds 1.1 and 2a.11.1; 1.1 and 2a.11.2; 1.1 and 2a.11.3; 1.1 and 2a.11.4; 1.1 and 2a.11.5; 1.1 and 2a.11.6; 1.2 and 2a.11.1; 1.2 and 2a.11.2; 1.2 and 2a.11.3; 1.2 and 2a.11.4; 1.2 and 2a.11.5; 1.2 and 2a.11.6; 1.3 and 2a.11.1; 1.3 and 2a.11.2; 1.3 and 2a.11.3; 1.3 and 2a.11.4; 1.3 and 2a.11.5; 1.3 and 2a.11.6; 1.4 and 2a.11.1; 1.4 and 2a.11.2; 1.4 and 2a.11.3; 1.4 and 2a.11.4; 1.4 and 2a.11.5; 1.4 and 2a.11.6; 1.5 and 2a.11.1; 1.5 and 2a.11.2; 1.5 and 2a.11.3; 1.5 and 2a.11.4; 1.5 and 2a.11.5; 1.5 and 2a.11.6; 1.6 and 2a.11.1; 1.6 and 2a.11.2; 1.6 and 2a.11.3; 1.6 and 2a.11.4; 1.6 and 2a.11.5; 1.6 and 2a.11.6; 1.7 and 2a.11.1; 1.7 and 2a.11.2; 1.7 and 2a.11.3; 1.7 and 2a.11.4; 1.7 and 2a.11.5; 1.7 and 2a.11.6; 1.12 and 2a.11.1; 1.12 and 2a.11.2; 1.12 and 2a.11.3; 1.12 and 2a.11.4; 1.12 and 2a.11.5; 1.12 and 2a.11.6; 1.14 and 2a.11.1; 1.14 and 2a.11.2; 1.14 and 2a.11.3; 1.14 and 2a.11.4; 1.14 and 2a.11.5; 1.14 and 2a.11.6; 1.15 and 2a.11.1; 1.15 and 2a.11.2; 1.15 and 2a.11.3; 1.15 and 2a.11.4; 1.15 and 2a.11.5; 1.15 and 2a.11.6, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.15. Also preferred, of the above-mentioned combinations according to the invention, are those which contain as compound 2a.11 one of the compounds 2a.11.2, 2a.11.4, 2a.11.5 or 2a.11.6, while those combinations which contain the compounds 2a.11.5 or 2a.11.6 are particularly important according to the invention.

In another preferred embodiment of the present invention the anticholinergics 2a contained in the medicament combinations according to the invention are selected from the compounds of formula 2a.12

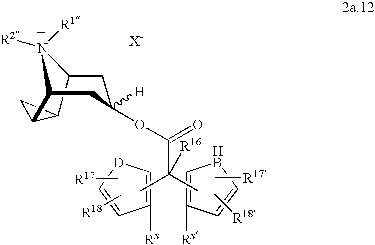

wherein X⁻ may have the meanings given above and wherein
D and B which may be identical or different, preferably identical, denote O, S, NH, CH₂, CH═CH or N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;

R^{1″} and R^{2″} which may be identical or different, denote —C₁-C₅-alkyl, which may optionally be substituted by —C₃-C₆-cycloalkyl, hydroxy or halogen, or R^{1″} and R^{2″} together denote a —C₃-C₅-alkylene bridge;
R¹⁷, R¹⁸, R¹⁷′ and R¹⁸′, which may be identical or different, denote hydrogen, —C₁-C₄-alkyl, —C₁-C₄-alkyloxy, hydroxy, —CF₃, —CHF₂, CN, NO₂ or halogen;
R^{x} and R^{x′} which may be identical or different denote hydrogen, —C₁-C₄-alkyl, —C₁-C₄-alkyloxy, hydroxy, —CF₃, —CHF₂, CN, NO₂ or halogen, or R^{x} and R^{x′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂.

The compounds of formula 2a.12 are known in the art (WO 03/064418).

Within the scope of the medicament combinations according to the invention preferred compounds of formula 2a.12 are those wherein
X⁻ denotes chloride, bromide or methanesulphonate, preferably bromide;
D and B which may be identical or different, preferably identical, denote O, S, NH or CH═CH;
R¹⁶ denotes hydrogen, hydroxy, —C₁-C₄-alkyl, —C₁-C₄-alkyloxy, —CF₃, —CHF₂, fluorine, chlorine or bromine;
R^{1″} and R^{2″} which may be identical or different, denote C₁-C₄-alkyl, which may optionally be substituted by hydroxy, fluorine, chlorine or bromine, or R^{1″} and R^{2″} together denote a —C₃-C₄-alkylene bridge;

R¹⁷, R¹³, R^{17′} and R^{18′}, which may be identical or different, denote hydrogen, C₁-C₄-alkyl, C₁-C₄-alkyloxy, hydroxy, —CF₃, —CHF₂, CN, NO₂, fluorine, chlorine or bromine;
R^{x} and R^{x′} which may be identical or different denote hydrogen, C₁-C₄-alkyl, C₁-C₄-alkyloxy, hydroxy, —CF₃, —CHF₂, CN, NO₂, fluorine, chlorine or bromine, or R^{x} and R^{x′} together denote a single bond or a double-bonded group selected from O, S, NH— and CH₂.

Within the scope of the medicament combinations according to the invention particularly preferred compounds of formula 2a.12 are those wherein
X⁻ denotes chloride, bromide, or methanesulphonate, preferably bromide;
D and B which may be identical or different, preferably identical, denote S or CH═CH;
R¹⁶ denotes hydrogen, hydroxy or methyl;
R^{1″} and R^{2″} which may be identical or different denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different denote hydrogen, —CF₃ or fluorine, preferably hydrogen, or R^{x} and R^{x′} together denote a single bond or —O.

Within the scope of the medicament combinations according to the invention particularly preferred compounds of formula 2a.12 are also those wherein
X⁻ denotes bromide;
D and B denote —CH═CH—;
R¹⁶ denotes hydrogen, hydroxy or methyl;
R^{1″} and R^{2″} denotes methyl;
R¹⁷, R¹⁸, R^{17′} and R^{18′}, which may be identical or different, denote hydrogen or fluorine, preferably hydrogen;
R^{x} and R^{x′} which may be identical or different denote hydrogen or fluorine, preferably hydrogen, or R^{x} and R^{x′} together denote a single bond or the group —O.

Of particular importance are those medicament combinations which contain in addition to a compound of formula 1 one of the following compounds of formula 2a.12:
cyclopropyltropine benzilate-methobromide (2a.12.1);
cyclopropyltropine 2,2-diphenylpropionate-methobromide (2a.12.2);
cyclopropyltropine 9-hydroxy-xanthene-9-carboxylate-methobromide (2a.12.3);
cyclopropyltropine 9-methyl-fluorene-9-carboxylate-methobromide (2a.12.4);
cyclopropyltropine 9-methyl-xanthene-9-carboxylate-methobromide (2a.12.5);
cyclopropyltropine 9-hydroxy-fluorene-9-carboxylate-methobromide (2a.12.6);
methyl cyclopropyltropine 4,4′-difluorobenzilate-methobromide (2a.12.7).

The compounds of formula 2a.12 may optionally be present in the form of the enantiomers, mixtures of enantiomers or racemates thereof, as well as optionally in the form of the hydrates and/or solvates thereof.

Examples of novel medicament combinations of preferred compounds of formula 1 with the above-mentioned anticholinergics 2a.12 are combinations containing the compounds 1.1 and 2a.12.1; 1.1 and 2a.12.2; 1.1 and 2a.12.3; 1.1 and 2a.12.4; 1.1 and 2a.12.5; 1.1 and 2a.12.6; 1.1 and 2a.12.7; 1.2 and 2a.12.1; 1.2 and 2a.12.2; 1.2 and 2a.12.3; 1.2 and 2a.12.4; 1.2 and 2a.12.5; 1.2 and 2a.12.6; 1.2 and 2a.12.7; 1.3 and 2a.12.1; 1.3 and 2a.12.2; 1.3 and 2a.12.3; 1.3 and 2a.12.4; 1.3 and 2a.12.5; 1.3 and 2a.12.6; 1.3 and 2a.12.7; 1.4 and 2a.12.1; 1.4 and 2a.12.2; 1.4 and 2a.12.3; 1.4 and 2a.12.4; 1.4 and 2a.12.5; 1.4 and 2a.12.6; 1.4 and 2a.12.7; 1.5 and 2a.12.1; 1.5 and 2a.12.2; 1.5 and 2a.12.3; 1.5 and 2a.12.4; 1.5 and 2a.12.5; 1.5 and 2a.12.6; 1.5 and 2a.12.7; 1.6 and 2a.12.1; 1.6 and 2a.12.2; 1.6 and 2a.12.3; 1.6 and 2a.12.4; 1.6 and 2a.12.5; 1.6 and 2a.12.6; 1.6 and 2a.12.7; 1.7 and 2a.12.1; 1.7 and 2a.12.2; 1.7 and 2a.12.3; 1.7 and 2a.12.4; 1.7 and 2a.12.5; 1.7 and 2a.12.6; 1.7 and 2a.12.7; 1.12 and 2a.12.1; 1.12 and 2a.12.2; 1.12 and 2a.12.3; 1.12 and 2a.12.4; 1.12 and 2a.12.5; 1.12 and 2a.12.6; 1.12 and 2a.12.7; 1.14 and 2a.12.1; 1.14 and 2a.12.2; 1.14 and 2a.12.3; 1.14 and 2a.12.4; 1.14 and 2a.12.5; 1.14 and 2a.12.6; 1.14 and 2a.12.7; 1.15 and 2a.12.1; 1.15 and 2a.12.2; 1.15 and 2a.12.3; 1.15 and 2a.12.4; 1.15 and 2a.12.5; 1.15 and 2a.12.6; 1.15 and 2a.12.7, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.12. Also preferred, of the above-mentioned combinations according to the invention, are those which contain as compound 2a.11 one of the compounds 2a.12.1, 2a.12.2, 2a.12.5 or 2a.12.7, while those combinations which contain the compounds 2a.12.1 or 2a.12.2 are particularly important according to the invention.

In another preferred embodiment of the present invention the anticholinergics 2a contained in the medicament combinations according to the invention are selected from the compounds of formula 2a.13

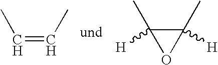

wherein X⁻ may have the meanings given above and wherein
A′ denotes a double-bonded group selected from

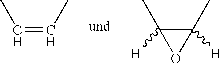

R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen, or
   R^{1′″} and R^{2′″} together denote a —C₃-C₅-alkylene bridge;
R²⁰, R²¹, R^{20′} and R^{21′} which may be identical or different denote hydrogen, —C₁-C₄-alkyl, —C₁-C₄-alkyloxy, hydroxy, —CF₃, —CHF₂, CN, NO₂ or halogen.

The compounds of formula 2a.13 are known in the art (WO 03/064417).

Within the scope of the medicament combinations according to the invention preferred compounds of formula 2a.13 are those wherein
A′ denotes a double-bonded group selected from

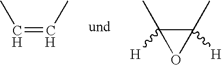

X⁻ denotes chloride, bromide or methanesulphonate, preferably bromide;
R¹⁹ denotes hydroxy or methyl;
R^{1′″} and R^{2′″} which may be identical or different denote methyl or ethyl, preferably methyl;
R²⁰, R²¹, R^{20′} and R^{21′} which may be identical or different denote hydrogen, —CF₃, —CHF₂ or fluorine, preferably hydrogen or fluorine.

Within the scope of the medicament combinations according to the invention particularly preferred compounds of formula 2a.13 are those wherein
A′ denotes a double-bonded group selected from

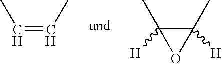

X⁻ denotes bromide;
R¹⁹ denotes hydroxy or methyl, preferably methyl;
R^{1′″} and R^{2′″} which may be identical or different denote methyl or ethyl, preferably methyl;
R³, R⁴, R^{3′} and R^{4′} which may be identical or different denote hydrogen or fluorine.

Of particular importance are those medicament combinations which contain in addition to a compound of formula 1 one of the following compounds of formula 2a.13:
tropenol 9-hydroxy-xanthene-9-carboxylate-methobromide (2a.13.1);
scopine 9-hydroxy-xanthene-9-carboxylate methobromide (2a.13.2);
tropenol 9-methyl-xanthene-9-carboxylate-methobromide (2a.13.3);
scopine 9-methyl-xanthene-9-carboxylate-methobromide (2a.13.4);
tropenol 9-ethyl-xanthene-9-carboxylate methobromide (2a.13.5);
tropenol 9-difluoromethyl-xanthene-9-carboxylate-methobromide (2a.13.6);
scopine 9-hydroxymethyl-xanthene-9-carboxylate-methobromide (2a.13.7).

The compounds of formula 2a.13 may optionally be present in the form of the enantiomers, mixtures of enantiomers or racemates thereof, as well as optionally in the form of the hydrates and/or solvates thereof.

Examples of novel medicament combinations of preferred compounds of formula 1 with the above-mentioned anticholinergics 2a.13 are combinations containing the compounds 1.1 and 2a.13.1; 1.1 and 2a.13.2; 1.1 and 2a.13.3; 1.1 and 2a.13.4; 1.1 and 2a.13.5; 1.1 and 2a.13.6; 1.1 and 2a.13.7; 1.2 and 2a.13.1; 1.2 and 2a.13.2; 1.2 and 2a.13.3; 1.2 and 2a.13.4; 1.2 and 2a.13.5; 1.2 and 2a.13.6; 1.2 and 2a.13.7; 1.3 and 2a.13.1; 1.3 and 2a.13.2; 1.3 and 2a.13.3; 1.3 and 2a.13.4; 1.3 and 2a.13.5; 1.3 and 2a.13.6; 1.3 and 2a.13.7; 1.4 and 2a.13.1; 1.4 and 2a.13.2; 1.4 and 2a.13.3; 1.4 and 2a.13.4; 1.4 and 2a.13.5; 1.4 and 2a.13.6; 1.4 and 2a.13.7; 1.5 and 2a.13.1; 1.5 and 2a.13.2; 1.5 and 2a.13.3; 1.5 and 2a.13.4; 1.5 and 2a.13.5; 1.5 and 2a.13.6; 1.5 and 2a.13.7; 1.6 and 2a.13.1; 1.6 and 2a.13.2; 1.6 and 2a.13.3; 1.6 and 2a.13.4; 1.6 and 2a.13.5; 1.6 and 2a.13.6; 1.6 and 2a.13.7; 1.7 and 2a.13.1; 1.7 and 2a.13.2; 1.7 and 2a.13.3; 1.7 and 2a.13.4; 1.7 and 2a.13.5; 1.7 and 2a.13.6; 1.7 and 2a.13.7; 1.12 and 2a.13.1; 1.12 and 2a.13.2; 1.12 and 2a.13.3; 1.12 and 2a.13.4; 1.12 and 2a.13.5; 1.12 and 2a.13.6; 1.12 and 2a.13.7; 1.14 and 2a.13.1; 1.14 and 2a.13.2; 1.14 and 2a.13.3; 1.14 and 2a.13.4; 1.14 and 2a.13.5; 1.14 and 2a.13.6; 1.14 and 2a.13.7; 1.15 and 2a.13.1; 1.15 and 2a.13.2; 1.15 and 2a.13.3; 1.15 and 2a.13.4; 1.15 and 2a.13.5; 1.15 and 2a.13.6; 1.15 and 2a.13.7, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.15. Also preferred, of the above-mentioned combinations according to the invention, are those which contain as compound 2a.11 one of the compounds 2a.13.2, 2a.13.3, 2a.13.4 or 2a.13.5, while those combinations which contain the compounds 2a.13.3 or 2a.13.4 are particularly important according to the invention.

Within the scope of the present invention any reference to anticholinergics 1′ is to be taken as being a reference to the pharmacologically active cations of the salts in question. These cations are tiotropium (2a.1′), oxitropium (2a.2′), flutropium (2a.3′), ipratropium (2a.4′), glycopyrronium (2a.5′), trospium (2a.6′) and the cations listed below:

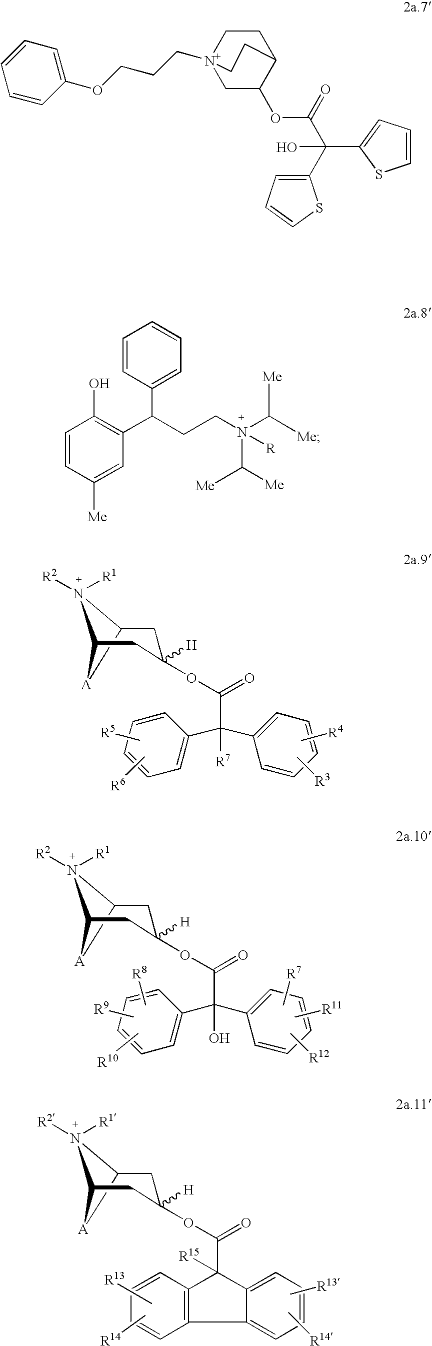

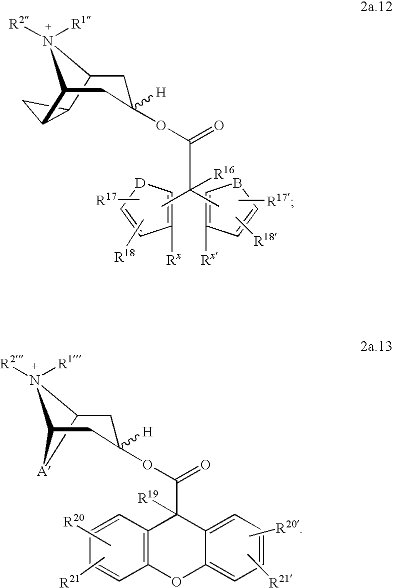

Other preferred medicament combinations according to the invention contain as a further active substance one or more, preferably one PDE IV inhibitor 2b in addition to one or more, preferably one compound of formula 1, optionally in combination with pharmaceutically acceptable excipients.

In medicament combinations of this kind the PDE IV inhibitor 2b is preferably selected from among enprofyllin, theophyllin, roflumilast, ariflo (cilomilast), CP-325,366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′—[N-2-cyano-S-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, C1-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3.4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof.

In particularly preferred medicament combinations the PDE IV inhibitor 2b is selected from among enprofyllin (2b.1), roflumilast (2b.2), ariflo (cilomilast) (2b.3), AWD-12-281 (GW-842470) (2b.4), N-(3,5-dichloro-1-oxo-pyridin-4-yl)-4-difluoromethoxy-3-cyclopropylmethoxybenzamide (2b.5), T-440 (2b.6), T-2585 (2b.7), arofyllin (2b.8), cis[4-cyano-4-(3-cyclopentyloxy-4-methoxyphenyl)cyclohexane-1-carboxylic acid] (2b.9), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol] (2b.11), PD-168787 (2b.12), atizoram (2b.13), V-11294A (2b.14), CI-1018 (2b.15), CDC-801 (2b.16), D-22888 (2b.17), YM-58997 (2b.18), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), optionally in the form of the racemates, enantiomers or diastereomers thereof and optionally in the form of the pharmacologically acceptable acid addition salts, solvates and/or hydrates thereof.

In particularly preferred medicament combinations the PDE IV inhibitor 2b is selected from among roflumilast (2b.2), ariflo (cilomilast) (2b.3), AWD-12-281 (GW-842470) (2b.4), arofyllin (2b.8), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol] (2b.11), atizoram (2b.13), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), while roflumilast (2b.2), Z-15370 (2b.19) and AWD-12-281 (2b.4) are of particular importance, optionally in the form of the racemates, enantiomers or diastereomers thereof and optionally in the form of the pharmacologically acceptable acid addition salts, solvates and/or hydrates thereof.

By acid addition salts with pharmacologically acceptable acids, which the compounds 2b are optionally capable of forming, are meant for example salts selected from among the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate.

Examples of novel preferred medicament combinations of compounds of formula 1 with the above-mentioned PDE IV-inhibitors 2b are combinations containing the compounds 1.1 and 2b.1; 1.1 and 2b.2; 1.1 and 2b.3; 1.1 and 2b.4; 1.1 and 2b.5; 1.1 and 2b.6; 1.1 and 2b.7; 1.1 and 2b.8; 1.1 and 2b.9; 1.1 and 2b.10; 1.1 and 2b.11; 1.1 and 2b.12; 1.1 and 2b.13; 1.1 and 2b.14; 1.1 and 2b.15; 1.1 and 2b.16; 1.1 and 2b.17; 1.1 and 2b.18; 1.1 and 2b.19; 1.1 and 2b.20; 1.1 and 2b.21; 1.2 and 2b.1; 1.2 and 2b.2; 1.2 and 2b.3; 1.2 and 2b.4; 1.2 and 2b.5; 1.2 and 2b.6; 1.2 and 2b.7; 1.2 and 2b.8; 1.2 and 2b.9; 1.2 and 2b.10; 1.2 and 2b.11; 1.2 and 2b.12; 1.2 and 2b.13; 1.2 and 2b.14; 1.2 and 2b.15; 1.2 and 2b.16; 1.2 and 2b.17; 1.2 and 2b.18; 1.2 and 2b.19; 1.2 and 2b.20; 1.2 and 2b.21; 1.3 and 2b.1; 1.3 and 2b.2; 1.3 and 2b.3; 1.3 and 2b.4; 1.3 and 2b.5; 1.3 and 2b.6; 1.3 and 2b.7; 1.3 and 2b.8; 1.3 and 2b.9; 1.3 and 2b.10; 1.3 and 2b.11; 1.3 and 2b.12; 1.3 and 2b.13; 1.3 and 2b.14; 1.3 and 2b.15; 1.3 and 2b.16; 1.3 and 2b.17; 1.3 and 2b.18; 1.3 and 2b.19; 1.3 and 2b.20; 1.3 and 2b.21; 1.4 and 2b.1; 1.4 and 2b.2; 1.4 and 2b.3; 1.4 and 2b.4; 1.4 and 2b.5; 1.4 and 2b.6; 1.4 and 2b.7; 1.4 and 2b.8; 1.4 and 2b.9; 1.4 and 2b.10; 1.4 and 2b.11; 1.4 and 2b.12; 1.4 and 2b.13; 1.4 and 2b.14; 1.4 and 2b.15; 1.4 and 2b.16; 1.4 and 2b.17; 1.4 and 2b.18; 1.4 and 2b.19; 1.4 and 2b.20; 1.4 and 2b.21; 1.5 and 2b.1; 1.5 and 2b.2; 1.5 and 2b.3; 1.5 and 2b.4; 1.5 and 2b.5; 1.5 and 2b.6; 1.5 and 2b.7; 1.5 and 2b.8; 1.5 and 2b.9; 1.5 and 2b.10; 1.5 and 2b.11; 1.5 and 2b.12; 1.5 and 2b.13; 1.5 and 2b.14; 1.5 and 2b.15; 1.5 and 2b.16; 1.5 and 2b.17; 1.5 and 2b.18; 1.5 and 2b.19; 1.5 and 2b.20; 1.5 and 2b.21; 1.6 and 2b.1; 1.6 and 2b.2; 1.6 and 2b.3; 1.6 and 2b.4; 1.6 and 2b.5; 1.6 and 2b.6; 1.6 and 2b.7; 1.6 and 2b.8; 1.6 and 2b.9; 1.6 and 2b.10; 1.6 and 2b.11; 1.6 and 2b.12; 1.6 and 2b.13; 1.6 and 2b.14; 1.6 and 2b.15; 1.6 and 2b.16; 1.6 and 2b.17; 1.6 and 2b.18; 1.6 and 2b.19; 1.6 and 2b.20; 1.6 and 2b.21; 1.7 and 2b.1; 1.7 and 2b.2; 1.7 and 2b.3; 1.7 and 2b.4; 1.7 and 2b.5; 1.7 and 2b.6; 1.7 and 2b.7; 1.7 and 2b.8; 1.7 and 2b.9; 1.7 and 2b.10; 1.7 and 2b.11; 1.7 and 2b.12; 1.7 and 2b.13; 1.7 and 2b.14; 1.7 and 2b.15; 1.7 and 2b.16; 1.7 and 2b.17; 1.7 and 2b.18; 1.7 and 2b.19; 1.7 and 2b.20; 1.7 and 2b.21; 1.12 and 2b.1; 1.12 and 2b.2; 1.12 and 2b.3; 1.12 and 2b.4; 1.12 and 2b.5; 1.12 and 2b.6; 1.12 and 2b.7; 1.12 and 2b.8; 1.12 and 2b.9; 1.12 and 2b.10; 1.12 and 2b.11; 1.12 and 2b.12; 1.12 and 2b.13; 1.12 and 2b.14; 1.12 and 2b.15; 1.12 and 2b.16; 1.12 and 2b.17; 1.12 and 2b.18; 1.12 and 2b.19; 1.12 and 2b.20; 1.12 and 2b.21; 1.14 and 2b.1; 1.14 and 2b.2; 1.14 and 2b.3; 1.14 and 2b.4; 1.14 and 2b.5; 1.14 and 2b.6; 1.14 and 2b.7; 1.14 and 2b.8; 1.14 and 2b.9; 1.14 and 2b.10; 1.14 and 2b.11; 1.14 and 2b.12; 1.14 and 2b.13; 1.14 and 2b.14; 1.14 and 2b.15; 1.14 and 2b.16; 1.14 and 2b.17; 1.14 and 2b.18; 1.14 and 2b.19; 1.14 and 2b.20; 1.14 and 2b.21; 1.15 and 2b.1; 1.15 and 2b.2; 1.15 and 2b.3; 1.15 and 2b.4; 1.15 and 2b.5; 1.15 and 2b.6; 1.15 and 2b.7; 1.15 and 2b.8; 1.15 and 2b.9; 1.15 and 2b.10; 1.15 and 2b.11; 1.15 and 2b.12; 1.15 and 2b.13; 1.15 and 2b.14; 1.15 and 2b.15; 1.15 and 2b.16; 1.15 and 2b.17; 1.15 and 2b.18; 1.15 and 2b.19; 1.15 and 2b.20 or 1.15 and 2b.21, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.15. Also preferred, of the above-mentioned combinations according to the invention, are those which contain as compound 2b one of the compounds 2b.2, 2b.3, 2b.4, 2b.8, 2b.10, 2b.11, 2b.13, 2b.19, 2b.20 or 2b.21, while those combinations which contain one of the compounds 2b.2, 2b.4 or 2b.19 are particularly important according to the invention.

Other preferred medicament combinations according to the invention contain as a further active substance one or more, preferably one steroid 2c in addition to one or more, preferably one, compound of formula 1, optionally in combination with pharmaceutically acceptable excipients.

In medicament combinations of this kind the steroid 2c is preferably selected from among prednisolone (2c.1), prednisone (2c.2), butixocortpropionate (2c.3), RPR-106541 (2c.4), flunisolide (2c.5), beclomethasone (2c.6), triamcinolone (2c.7), budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate (2c.16) and etiprednol-dichloroacetate (BNP-166, 2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from among flunisolide (2c.5), beclomethasone (2c.6), triamcinolone (2c.7), budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate (2c.16) and etiprednol-dichloroacetate (2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from among budesonide (2c.8), fluticasone (2c.9), mometasone (2c.10), ciclesonide (2c.11), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15) and etiprednol-dichloroacetate (2c.17), optionally in the form of the racemates, enantiomers or diastereomers thereof and optionally in the form of the salts and derivatives thereof, the solvates and/or hydrates thereof.

Any reference to steroids 2c includes a reference to any salts or derivatives, hydrates or solvates thereof which may exist. Examples of possible salts and derivatives of the steroids 2c may be: alkali metal salts, such as for example sodium or potassium salts, sulphobenzoates, phosphates, isonicotinates, acetates, propionates, dihydrogen phosphates, palmitates, pivalates or also furoates.

Examples of novel preferred medicament combinations of preferred compounds of formula 1 with the above-mentioned steroids 2c are combinations containing the compounds 1.1 and 2c.1; 1.1 and 2c.2; 1.1 and 2c.3; 1.1 and 2c.4; 1.1 and 2c.5; 1.1 and 2c.6; 1.1 and 2c.7; 1.1 and 2c.8; 1.1 and 2c.9; 1.1 and 2c.10; 1.1 and 2c.11; 1.1 and 2c.12; 1.1 and 2c.13; 1.1 and 2c.14; 1.1 and 2c.15; 1.1 and 2c.16; 1.1 and 2c.17; 1.2 and 2c.1; 1.2 and 2c.2; 1.2 and 2c.3; 1.2 and 2c.4; 1.2 and 2c.5; 1.2 and 2c.6; 1.2 and 2c7; 1.2 and 2c.8; 1.2 and 2c.9; 1.2 and 2c.10; 1.2 and 2c.11; 1.2 and 2c.12; 1.2 and 2c.13; 1.2 and 2c.14; 1.2 and 2c.15; 1.2 and 2c.16; 1.2 and 2c.17; 1.3 and 2c.1; 1.3 and 2c.2; 1.3 and 2c.3; 1.3 and 2c.4; 1.3 and 2c.5; 1.3 and 2c.6; 1.3 and 2c.7; 1.3 and 2c.8; 1.3 and 2c.9; 1.3 and 2c.10; 1.3 and 2c.11; 1.3 and 2c.12; 1.3 and 2c.13; 1.3 and 2c.14; 1.3 and 2c.15; 1.3 and 2c.16; 1.3 and 2c.17; 1.4 and 2c.1; 1.4 and 2c.2; 1.4 and 2c.3; 1.4 and 2c.4; 1.4 and 2c.5; 1.4 and 2c.6; 1.4 and 2c.7; 1.4 and 2c.8; 1.4 and 2c.9; 1.4 and 2c.10; 1.4 and 2c.11; 1.4 and 2c.12; 1.4 and 2c.13; 1.4 and 2c.14; 1.4 and 2c.15; 1.4 and 2c.16; 1.4 and 2c.17; 1.5 and 2c.1; 1.5 and 2c.2; 1.5 and 2c.3; 1.5 and 2c.4; 1.5 and 2c.5; 1.5 and 2c.6; 1.5 and 2c.7; 1.5 and 2c.8; 1.5 and 2c.9; 1.5 and 2c.10; 1.5 and 2c.11; 1.5 and 2c.12; 1.5 and 2c.13; 1.5 and 2c.14; 1.5 and 2c.15; 1.5 and 2c.16; 1.5 and 2c.17; 1.6 and 2c.1; 1.6 and 2c.2; 1.6 and 2c.3; 1.6 and 2c.4; 1.6 and 2c.5; 1.6 and 2c.6; 1.6 and 2c.7; 1.6 and 2c.8; 1.6 and 2c.9; 1.6 and 2c.10; 1.6 and 2c.11; 1.6 and 2c.12; 1.6 and 2c.13; 1.6 and 2c.14; 1.6 and 2c.15; 1.6 and 2c.16; 1.6 and 2c.17; 1.7 and 2c.1; 1.7 and 2c.2; 1.7 and 2c.3; 1.7 and 2c.4; 1.7 and 2c.5; 1.7 and 2c.6; 1.7 and 2c.7; 1.7 and 2c.8; 1.7 and 2c.9; 1.7 and 2c.10; 1.7 and 2c.11; 1.7 and 2c.12; 1.7 and 2c.13; 1.7 and 2c.14; 1.7 and 2c.15; 1.7 and 2c.16; 1.7 and 2c.17; 1.12 and 2c.1; 1.12 and 2c.2; 1.12 and 2c.3; 1.12 and 2c.4; 1.12 and 2c.5; 1.12 and 2c.6; 1.12 and 2c.7; 1.12 and 2c.8; 1.12 and 2c.9; 1.12 and 2c.10; 1.12 and 2c.11; 1.12 and 2c.12; 1.12 and 2c.13; 1.12 and 2c.14; 1.12 and 2c.15; 1.12 and 2c.16; 1.12 and 2c.17; 1.14 and 2c.1; 1.14 and 2c.2; 1.14 and 2c.3; 1.14 and 2c.4; 1.14 and 2c.5; 1.14 and 2c.6; 1.14 and 2c.7; 1.14 and 2c.8; 1.14 and 2c.9; 1.14 and 2c.10; 1.14 and 2c.11; 1.14 and 2c.12; 1.14 and 2c.13; 1.14 and 2c.14; 1.14 and 2c.15; 1.14 and 2c.16; 1.14 and 2c.17; 1.15 and 2c.1; 1.15 and 2c.2; 1.15 and 2c.3; 1.15 and 2c.4; 1.15 and 2c.5; 1.15 and 2c.6; 1.15 and 2c.7; 1.15 and 2c.8; 1.15 and 2c.9; 1.15 and 2c.10; 1.15 and 2c.11; 1.15 and 2c.12; 1.15 and 2c.13; 1.15 and 2c.14; 1.15 and 2c.15; 1.15 and 2c.16 or 1.15 and 2c.17 in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.15. Also preferred, of the above-mentioned combinations according to the invention, are those which contain as compound 2c one of the compounds 2c.5, 2c.6, 2c.7, 2c.8, 2c.9, 2c.10, 2c.11, 2c.12, 2c.13, 2c.14, 2c.15, 2c.16 or 2c.17, while those combinations that contain one of the compounds 2c.8, 2c.9, 2c.10, 2c.11, 2c.15 or 2c.17 are particularly important according to the invention.

Other preferred medicament combinations according to the invention contain, as an additional active substance, one or more, preferably one, LTD4 antagonist 2d in addition to one or more, preferably one compound of formula 1, optionally in combination with pharmaceutically acceptable excipients.

In such medicament combinations the LTD4 antagonist 2d is preferably selected from among montelukast (2d.1), 1-(((R)-(3-(2-(6,7-difluoro-2-quinolinypethenyl)phenyl)-3-(2-(2-hydroxy-2-propyl)phenyl)thio)methylcyclopropane-acetic acid (2d.2), 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropanacetic acid (2d.3), pranlukast (2d.4), zafirlukast (2d.5), [2-[[2-(4-tert-butyl-2-thiazolyl)-5-benzofuranyl]oxymethyl]-phenyl]acetic acid (2d.6), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In preferred medicament combinations the LTD4 antagonist 2d is selected from the group comprising montelukast (2d.1), pranlukast (2d.4), zafirlukast (2d.5), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In particularly preferred medicament combinations the LTD4 antagonist 2d is selected from the group comprising montelukast (2d.1), pranlukast (2d.4), zafirlukast (2d.5), MCC-847 (ZD-3523) (2d.7), MN-001 (2d.8) and MEN-91507 (LM-1507) (2d.9), while montelukast (2d.1), pranlukast (2d.4) and zafirlukast (2d.5) are particularly preferred, optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

By the acid addition salts with pharmacologically acceptable acids which the compounds 2d may possibly be capable of forming are meant for example salts selected from the group comprising the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably the hydrochloride, hydrobromide, hydrosulphate, hydrophosphate, hydrofumarate and hydromethanesulphonate. Examples of possible salts and derivatives which the compounds 2d may possibly be capable of forming include for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

Examples of novel preferred medicament combinations of preferred compounds of formula 1 with the above-mentioned LTD4-antagonists 2d are combinations containing the compounds 1.1 and 2d.1; 1.1 and 2d.2; 1.1 and 2d.3; 1.1 and 2d.4; 1.1 and 2d.5; 1.1 and 2d.6; 1.1 and 2d.7; 1.1 and 2d.8; 1.1 and 2d.9; 1.1 and 2d.10; 1.1 and 2d.11; 1.1 and 2d.12; 1.2 and 2d.1; 1.2 and 2d.2; 1.2 and 2d.3; 1.2 and 2d.4; 1.2 and 2d.5; 1.2 and 2d.6; 1.2 and 2d.7; 1.2 and 2d.8; 1.2 and 2d.9; 1.2 and 2d.10; 1.2 and 2d.11; 1.2 and 2d.12; 1.3 and 2d.1; 1.3 and 2d.2; 1.3 and 2d.3; 1.3 and 2d.4; 1.3 and 2d.5; 1.3 and 2d.6; 1.3 and 2d.7; 1.3 and 2d.8; 1.3 and 2d.9; 1.3 and 2d.10; 1.3 and 2d.11; 1.3 and 2d.12; 1.4 and 2d.1; 1.4 and 2d.2; 1.4 and 2d.3; 1.4 and 2d.4; 1.4 and 2d.5; 1.4 and 2d.6; 1.4 and 2d.7; 1.4 and 2d.8; 1.4 and 2d.9; 1.4 and 2d.10; 1.4 and 2d.11; 1.4 and 2d.12; 1.5 and 2d.1; 1.5 and 2d.2; 1.5 and 2d.3; 1.5 and 2d.4; 1.5 and 2d.5; 1.5 and 2d.6; 1.5 and 2d.7; 1.5 and 2d.8; 1.5 and 2d.9; 1.5 and 2d.10; 1.5 and 2d.11; 1.5 and 2d.12; 1.6 and 2d.1; 1.6 and 2d.2; 1.6 and 2d.3; 1.6 and 2d.4; 1.6 and 2d.5; 1.6 and 2d.6; 1.6 and 2d.7; 1.6 and 2d.8; 1.6 and 2d.9; 1.6 and 2d.10; 1.6 and 2d.11; 1.6 and 2d.12; 1.7 and 2d.1; 1.7 and 2d.2; 1.7 and 2d.3; 1.7 and 2d.4; 1.7 and 2d.5; 1.7 and 2d.6; 1.7 and 2d.7; 1.7 and 2d.8; 1.7 and 2d.9; 1.7 and 2d.10; 1.7 and 2d.11; 1.7 and 2d.12; 1.12 and 2d.1; 1.12 and 2d.2; 1.12 and 2d.3; 1.12 and 2d.4; 1.12 and 2d.5; 1.12 and 2d.6; 1.12 and 2d.7; 1.12 and 2d.8; 1.12 and 2d.9; 1.12 and 2d.10; 1.12 and 2d.11; 1.12 and 2d.12; 1.14 and 2d.1; 1.14 and 2d.2; 1.14 and 2d.3; 1.14 and 2d.4; 1.14 and 2d.5; 1.14 and 2d.6; 1.14 and 2d.7; 1.14 and 2d.8; 1.14 and 2d.9; 1.14 and 2d.10; 1.14 and 2d.11; 1.14 and 2d.12; 1.15 and 2d.1; 1.15 and 2d.2; 1.15 and 2d.3; 1.15 and 2d.4; 1.15 and 2d.5; 1.15 and 2d.6; 1.15 and 2d.7; 1.15 and 2d.8; 1.15 and 2d.9; 1.15 and 2d.10; 1.15 and 2d.11 or 1.15 and 2d.12, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.15. Also preferred, of the above-mentioned combinations according to the invention, are those which contain as compound 2d one of the compounds 2d.1, 2d.4, 2d.5, 2d.7, 2d.8, 2d.9, 2d.10, 2d.11 or 2d.12, while those combinations that contain one of the compounds 2d.1, 2d.4, 2d.5, 2d.7, 2d.8 or 2d.9 are particularly important according to the invention, while exceptional importance attaches to those combinations which contain one of the compounds 2d.1, 2d.4 or 2d.5.

Other preferred medicament combinations according to the invention contain one or more, preferably one, EGFR-inhibitor 2e as an additional active substance in addition to one or more, preferably one compound of formula 1, optionally in combination with pharmaceutically acceptable excipients.

In such medicament combinations the EGFR-inhibitor 2e is selected for example from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy- quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2.6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, cetuximab, trastuzumab, ABX-EGF and Mab ICR-62, optionally in the form of the racemates, enantiomers or diastereomers thereof, optionally in the form of the pharmacologically acceptable acid addition salts thereof, the solvates and/or hydrates thereof.

In such medicament combinations the EGFR-inhibitor 2e is preferably selected from among 4-[(3-chloro-4-fluorophenyl)amino]-6-{[4-(morpholin-4-yl)-1-oxo-2-buten-1-yl]-amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2.6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and cetuximab, optionally in the form of the racemates, enantiomers or diastereomers thereof, optionally in the form of the pharmacologically acceptable acid addition salts thereof, the solvates and/or hydrates thereof.

Particularly preferably, the EGFR-inhibitors 2a used within the scope of the medicament combinations according to the invention are selected from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-ethynyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and 4-[(3-chloro-4-fluoro-phenyl)amino]-6-{1-[(2-methoxyethyl)carbonyl]-piperidin-4-yloxy}-7-methoxy-quinazoline, optionally in the form of the racemates, enantiomers or diastereomers thereof, optionally in the form of the pharmacologically acceptable acid addition salts thereof, the solvates and/or hydrates thereof.

Particularly preferred medicament combinations according to the invention contain as EGFR-inhibitors 2e those compounds which are selected from the group comprising
4-[(3-chloro-4-fluorophenyl)amino]-6-{[4-(morpholin-4-yl)-1-oxo-2-buten-1-yl]amino}-7-cyclopropylmethoxy-quinazoline (2e.1),
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline (2e.2),
4-[(3-chloro-4-fluoro-phenyl)amino]-6-[2-((S)-6-methyl-2-oxo-morpholin-4-yl)-ethoxy]-7-methoxy-quinazoline (2e.3),
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline (2e.4),
4-[(3-ethynyl-phenyl)amino]-6,7-bis-(2-methoxy-ethoxy)-quinazoline (2e.5),
4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline (2e.6),
4-[(3-ethynyl-phenyl)amino]-6-{[4-(5,5-dimethyl-2-oxo-morpholin-4-yl)-1-oxo-2-buten-1-yl]amino}-quinazoline (2e.7),
4-[(3-chloro-4-fluoro-phenyl)amino]-6-(trans-4-methanesulphonylamino-cyclohexan-1-yloxy)-7-methoxy-quinazoline (2e.8),
4-[(3-chloro-4-fluoro-phenyl)amino]-6-(tetrahydropyran-3-yloxy)-7-methoxy-quinazoline (2e.9),
4-[(3-chloro-4-fluoro-phenyl)amino]-6-{1-[(morpholin-4-yl)carbonyl]-piperidin-4-yloxy}-7-methoxy-quinazoline (2e.10),
4-[(3-chloro-4-fluoro-phenyl)amino]-6-{1-[2-(2-oxopyrrolidin-1-yl)ethyl]-piperidin-4-yloxy}-7-methoxy-quinazoline (2e.11),
4-[(3-ethynyl-phenyl)amino]-6-(1-acetyl-piperidin-4-yloxy)-7-methoxy-quinazoline (2e.12),
4-[(3-ethynyl-phenyl)amino]-6-(1-methyl-piperidin-4-yloxy)-7-methoxy-quinazoline (2e.13),
4-[(3-ethynyl-phenyl)amino]-6-(1-methanesulphonyl-piperidin-4-yloxy)-7-methoxy-quinazoline (2e.14),
4-[(3-ethynyl-phenyl)amino]-6-{1-[(morpholin-4-yl)carbonyl]-piperidin-4-yloxy}-7-methoxy-quinazoline (2e.15),
4-[(3-chloro-4-fluoro-phenyl)amino]-6-{1-[(2-methoxyethyl)carbonyl]-piperidin-4-yloxy}-7-methoxy-quinazoline (2e.16),
4-[(3-chloro-4-fluoro-phenyl)amino]-6-[cis-4-(N-methanesulphonyl-N-methyl-amino)-cyclohexan-1-yloxy]-7-methoxy-quinazoline (2e.17),
4-[(3-chloro-4-fluoro-phenyl)amino]-6-[cis-4-(N-acetyl-N-methyl-amino)-cyclohexan-1-yloxy]-7-methoxy-quinazoline (2e.18),
4-[(3-chloro-4-fluoro-phenyl)amino]-6-(trans-4-methylamino-cyclohexan-1-yloxy)-7-methoxy-quinazoline (2e.19),
4-[(3-chloro-4-fluoro-phenyl)amino]-6-[trans-4-(N-methanesulphonyl-N-methyl-amino)-cyclohexan-1-yloxy]-7-methoxy-quinazoline (2e.20),
4-[(3-chloro-4-fluoro-phenyl)amino]-6-(trans-4-dimethylamino-cyclohexan-1-yloxy)-7-methoxy-quinazoline (2e.21),
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline (2e.22),
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline (2e.23),
4-[(3-chloro-4-fluoro-phenyl)amino]-6-(1-methanesulphonyl-piperidin-4-yloxy)-7-methoxy-quinazoline (2e.24) and
4-[(3-chloro-4-fluoro-phenyl)amino]-6-(1-cyano-piperidin-4-yloxy)-7-methoxy-quinazoline (2e.25),

optionally in the form of the racemates, enantiomers or diastereomers thereof, optionally in the form of the pharmacologically acceptable acid addition salts thereof, the solvates and/or hydrates thereof.

By the acid addition salts with pharmacologically acceptable acids which the compounds 2e may possibly be capable of forming are meant for example salts selected from the group comprising the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of novel preferred medicament combinations of preferred compounds of formula 1 with the above-mentioned EGFR-inhibitors 2e are combinations containing the compounds 1.1 and 2e.1; 1.1 and 2e.2; 1.1 and 2e.3; 1.1 and 2e.4; 1.1 and 2e.5; 1.1 and 2e.6; 1.1 and 2e.7; 1.1 and 2e.8; 1.1 and 2e.9; 1.1 and 2e.10; 1.1 and 2e.11; 1.1 and 2e.12; 1.1 and 2e.13; 1.1 and 2e.14; 1.1 and 2e.15; 1.1 and 2e.16; 1.1 and 2e.17; 1.1 and 2e.18; 1.1 and 2e.19; 1.1 and 2e.20; 1.1 and 2e.21; 1.1 and 2e.22; 1.1 and 2e.23; 1.1 and 2e.24; 1.1 and 2e.25; 1.2 and 2e.1; 1.2 and 2e.2; 1.2 and 2e.3; 1.2 and 2e.4; 1.2 and 2e.5; 1.2 and 2e.6; 1.2 and 2e.7; 1.2 and 2e.8; 1.2 and 2e.9; 1.2 and 2e.10; 1.2 and 2e.11; 1.2 and 2e.12; 1.2 and 2e.13; 1.2 and 2e.14; 1.2 and 2e.15; 1.2 and 2e.16; 1.2 and 2e.17; 1.2 and 2e.18; 1.2 and 2e1.9; 1.2 and 2e.20; 1.2 and 2e.21; 1.2 and 2e.22; 1.2 and 2e.23; 1.2 and 2e.24; 1.2 and 2e.25; 1.3 and 2e.1; 1.3 and 2e.2; 1.3 and 2e.3; 1.3 and 2e.4; 1.3 and 2e.5; 1.3 and 2e.6; 1.3 and 2e.7; 1.3 and 2e.8; 1.3 and 2e.9; 1.3 and 2e.10; 1.3 and 2e.11; 1.3 and 2e.12; 1.3 and 2e.13; 1.3 and 2e.14; 1.3 and 2e.15; 1.3 and 2e.16; 1.3 and 2e.17; 1.3 and 2e.18; 1.3 and 2e.19; 1.3 and 2e.20; 1.3 and 2e.21; 1.3 and 2e.22; 1.3 and 2e.23; 1.3 and 2e.24; 1.3 and 2e.25; 1.4 and 2e.1; 1.4 and 2e.2; 1.4 and 2e.3; 1.4 and 2e.4; 1.4 and 2e.5; 1.4 and 2e.6; 1.4 and 2e.7; 1.4 and 2e.8; 1.4 and 2e.9; 1.4 and 2e.10; 1.4 and 2e.11; 1.4 and 2e.12; 1.4 and 2e.13; 1.4 and 2e.14; 1.4 and 2e.15; 1.4 and 2e.16; 1.4 and 2e.17; 1.4 and 2e.18; 1.4 and 2e.19; 1.4 and 2e.20; 1.4 and 2e.21; 1.4 and 2e.22; 1.4 and 2e.23; 1.4 and 2e.24; 1.4 and 2e.25; 1.5 and 2e.1; 1.5 and 2e.2; 1.5 and 2e.3; 1.5 and 2e.4; 1.5 and 2e.5; 1.5 and 2e.6; 1.5 and 2e.7; 1.5 and 2e.8; 1.5 and 2e.9; 1.5 and 2e.10; 1.5 and 2e.11; 1.5 and 2e.12; 1.5 and 2e.13; 1.5 and 2e.14; 1.5 and 2e.15; 1.5 and 2e.16; 1.5 and 2e.17; 1.5 and 2e.18; 1.5 and 2e.19; 1.5 and 2e.20; 1.5 and 2e.21; 1.5 and 2e.22; 1.5 and 2e.23; 1.5 and 2e.24; 1.5 and 2e.25; 1.6 and 2e.1; 1.6 and 2e.2; 1.6 and 2e.3; 1.6 and 2e.4; 1.6 and 2e.5; 1.6 and 2e.6; 1.6 and 2e.7; 1.6 and 2e.8; 1.6 and 2e.9; 1.6 and 2e.10; 1.6 and 2e.11; 1.6 and 2e.12; 1.6 and 2e.13; 1.6 and 2e.14; 1.6 and 2e.15; 1.6 and 2e.16; 1.6 and 2e.17; 1.6 and 2e.18; 1.6 and 2e.19; 1.6 and 2e.20; 1.6 and 2e.21; 1.6 and 2e.22; 1.6 and 2e.23; 1.6 and 2e.24; 1.6 and 2e.25; 1.7 and 2e.1; 1.7 and 2e.2; 1.7 and 2e.3; 1.7 and 2e.4; 1.7 and 2e.5; 1.7 and 2e.6; 1.7 and 2e.7; 1.7 and 2e.8; 1.7 and 2e.9; 1.7 and 2e.10; 1.7 and 2e.11; 1.7 and 2e.12; 1.7 and 2e.13; 1.7 and 2e.14; 1.7 and 2e.15; 1.7 and 2e.16; 1.7 and 2e.17; 1.7 and 2e.18; 1.7 and 2e.19; 1.7 and 2e.20; 1.7 and 2e.21; 1.7 and 2e.22; 1.7 and 2e.23; 1.7 and 2e.24; 1.7 and 2e.25; 1.12 and 2e.1; 1.12 and 2e.2; 1.12 and 2e.3; 1.12 and 2e.4; 1.12 and 2e.5; 1.12 and 2e.6; 1.12 and 2e.7; 1.12 and 2e.8; 1.12 and 2e.9; 1.12 and 2e.10; 1.12 and 2e.11; 1.12 and 2e.12; 1.12 and 2e.13; 1.12 and 2e.14; 1.12 and 2e.15; 1.12 and 2e.16; 1.12 and 2e.17; 1.12 and 2e.18; 1.12 and 2e.19; 1.12 and 2e.20; 1.12 and 2e.21; 1.12 and 2e.22; 1.12 and 2e.23; 1.12 and 2e.24; 1.12 and 2e.25; 1.14 and 2e.1; 1.14 and 2e.2; 1.14 and 2e.3; 1.14 and 2e.4; 1.14 and 2e.5; 1.14 and 2e.6; 1.14 and 2e.7; 1.14 and 2e.8; 1.14 and 2e.9; 1.14 and 2e.10; 1.14 and 2e.11; 1.14 and 2e.12; 1.14 and 2e.13; 1.14 and 2e.14; 1.14 and 2e.15; 1.14 and 2e.16; 1.14 and 2e.17; 1.14 and 2e.18; 1.14 and 2e.19; 1.14 and 2e.20; 1.14 and 2e.21; 1.14 and 2e.22; 1.14 and 2e.23; 1.14 and 2e.24; 1.14 and 2e.25; 1.15 and 2e.1; 1.15 and 2e.2; 1.15 and 2e.3; 1.15 and 2e.4; 1.15 and 2e.5; 1.15 and 2e.6; 1.15 and 2e.7; 1.15 and 2e.8; 1.15 and 2e.9; 1.15 and 2e.10; 1.15 and 2e.11; 1.15 and 2e.12; 1.15 and 2e.13; 1.15 and 2e.14; 1.15 and 2e.15; 1.15 and 2e.16, 1.15 and 2e.17; 1.15 and 2e.18; 1.15 and 2e.19; 1.15 and 2e.20; 1.15 and 2e.21; 1.15 and 2e.22; 1.15 and 2e.23; 1.15 and 2e.24 or 1.15 and 2e.25, in each case optionally in the form of the racemates, enantiomers or diastereomers thereof and optionally in the form of the pharmacologically acceptable acid addition salts, solvates and/or hydrates thereof.

Of the above-mentioned combinations the preferred ones according to the invention are those which contain as compound of formula 1 one of the compounds 1.2, 1.5, 1.8, 1.10, 1.12 or 1.15. Also preferred, of the above-mentioned combinations according to the invention, are those which contain as compound 2e one of the compounds 2e.1, 2e.2, 2e.3, 2e.4, 2e.10, 2e.11, 2e.14, 2e.16, 2e.17, 2e.18, 2e.19, 2e.20, 2e.21, 2e.22, 2e.23, 2e.24 or 2e.25, while those combinations that contain one of the compounds 2e.2, 2e.3 or 2e.4 are particularly important according to the invention.

The novel medicament combinations comprising compounds of formula 1 with at least one other active substance 2 are not restricted to binary combinations of active substances. The combinations mentioned above, partly by way of example, which contain in addition to a compound of formula 1 one other active substance 2, may also contain a third or fourth, preferably a third active substance, which is also selected from the above-mentioned group of anticholinergics (2a), PDE-IV inhibitors (2b), steroids (2c), LTD4-antagonists (2d) and EGFR-inhibitors (2e).

Particularly preferred combinations which contain two other active substances in addition to a compound of formula 1 are selected from the active substance combinations listed below. These are medicament combinations which may contain, for example:

A) a compound of formula 1, an anticholinergic (2a), a PDEIV inhibitor (2b);

B) a compound of formula 1, an anticholinergic (2a), a steroid (2c);

C) a compound of formula 1, an anticholinergic (2a), an LTD4 antagonist (2d);

D) a compound of formula 1, an anticholinergic (2a), an EGFR inhibitor (2e);

E) a compound of formula 1, a PDEIV inhibitor (2b), a steroid (2c);

F) a compound of formula 1, a PDEIV inhibitor (2b), an LTD4 antagonist (2d);

G) a compound of formula 1, a PDEIV inhibitor (2b), an EGFR inhibitor (2e);

H) a compound of formula 1, a steroid (2c), an LTD4 antagonist (2d);

I) a compound of formula 1, a steroid (2c), an EGFR inhibitor (2e);

J) a compound of formula 1, an LTD4 antagonist (2d), an EGFR inhibitor (2e).

Of outstanding importance according to the invention are all those medicament combinations disclosed within the scope of the present invention which contain the compounds of formula 1 in the form of the R-enantiomers thereof.

Unless otherwise stated, the alkyl groups are straight-chained or branched alkyl groups having 1 to 4 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc.

Unless otherwise stated, the cycloalkyl groups are alicyclic groups with 3 to 6 carbon atoms. They are the cyclopropyl, cyclobutyl, cyclopentyl and cyclohexyl groups. Cyclopropyl is particularly important within the scope of the present invention.

Unless otherwise stated, the alkylene groups are branched and unbranched double-bonded alkyl bridges with 1 to 4 carbon atoms. Examples include: methylene, ethylene, propylene or butylene.

Unless otherwise stated, the alkylene-halogen groups are branched and unbranched double-bonded alkyl bridges with 1 to 4 carbon atoms which are mono-, di- or trisubstituted, preferably disubstituted, by a halogen. Accordingly, unless otherwise stated, alkylene-OH-groups are branched and unbranched double-bonded alkyl bridges with 1 to 4 carbon atoms which are mono-, di- or trisubstituted, preferably monosubstituted, by a hydroxy.

Unless otherwise stated, the term alkyloxy groups denotes branched and unbranched alkyl groups with 1 to 4 carbon atoms which are linked via an oxygen atom. Examples include: methyloxy, ethyloxy, propyloxy or butyloxy. In some cases the abbreviations MeO, EtO, PropO or BuO may be used to denote the methyloxy, ethyloxy, propyloxy or butyloxy groups. Unless otherwise stated, the definitions propyloxy and butyloxy include all the possible isomeric forms of the groups in question. Thus, for example, propyloxy includes n-propyloxy and iso-propyloxy, butyloxy includes iso-butyloxy, sec.butyloxy and tert.-butyloxy, etc. In some cases the term alkoxy may be used instead of alkyloxy within the scope of the present invention. The groups methyloxy, ethyloxy, propyloxy or butyloxy may therefore also be referred to by the names methoxy, ethoxy, propoxy or butoxy.

Unless otherwise stated, the term alkylene-alkyloxy refers to branched and unbranched double-bonded alkyl bridges with 1 to 4 carbon atoms which are mono-, di- or trisubstituted, preferably monosubstituted, by an alkyloxy group.

Unless otherwise stated, the term —O—CO-alkyl groups refers to branched and unbranched alkyl groups with 1 to 4 carbon atoms which are linked by an ester group. The alkyl groups are attached directly to the carbonyl carbon of the ester group. The term —O—CO-alkyl-halogen should be understood analogously. The group —O—CO—CF₃ denotes trifluoroacetate.

Halogen within the scope of the present invention denotes fluorine, chlorine, bromine or iodine. Unless stated otherwise, fluorine and bromine are the preferred halogens. The group CO denotes a carbonyl group.

Within the scope of the present invention, by a medicament combination of components 1 and 2 is meant the joint administration of both active substances in a single preparation or formulation or the separate administration of the two active substances in separate formulations. If the active substances 1 and 2 are administered in separate formulations, this separate administration may be done simultaneously or at different times, i.e. successively.

In one aspect the present invention relates to the above-mentioned medicament combinations which contain in addition to therapeutically effective amounts of 1 and 2 a pharmaceutically acceptable carrier. In one aspect the present invention relates to the above-mentioned pharmaceutical compositions which do not contain a pharmaceutically acceptable carrier in addition to therapeutically effective amounts of 1 and 2.

The present invention also relates to the use of therapeutically effective amounts of the active substances 1 for preparing a pharmaceutical composition also containing one or more, preferably one active substance 2 for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation.

In a preferred aspect the present invention relates to the use of therapeutically effective amounts of the active substance 1 for preparing a pharmaceutical composition also containing one or more, preferably one, active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD (chronic obstructive pulmonary disease) or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

The present invention also relates to the use of therapeutically effective amounts of an active substance of formula 1 in combination with therapeutically effective amounts of an active substance 2 for preparing a pharmaceutical composition for the treatment of one of the above-mentioned diseases.

The present invention also relates to a process for treating one of the above-mentioned diseases, which is characterised in that therapeutically effective amounts of active substance of formula 1 are administered in combination with therapeutically effective amounts of active substance 2.

Within the scope of the medicament combinations according to the invention, for example, 0.1-1000 μg of a compound of formula 1 may be administered per single dose. Preferably, 1-500 μg, particularly preferably 3-100 μg of the compound of formula 1 are administered per single dose, while a dosage range of from 5-75 μg, preferably from 7-50 μg is preferred according to the invention. Particularly preferably, the pharmaceutical compositions according to the invention are administered in an amount such that 9-40 μg, particularly preferably 11-30 μg, more preferably 12-25 μg of the compound of formula 1 are administered per single dose. For example, and without restricting the present invention thereto, 5 μg, 7.5 μg, 10 μg, 12.5 μg, 15 μg, 17.5 μg, 20 μg, 22.5 μg, 25 μg, 27.5 μg, 30 μg, 32.5 μg, 35 μg, 37.5 μg, 40 μg, 42.5 μg, 45 μg, 47.5 μg, 50 μg, 52.5 μg, 55 μg, 57.5 μg, 60 μg, 62.5 μg, 65 μg, 67.5 μg, 70 μg, 72.5 μg or 75 μg of a compound of formula 1 may be administered per single dose.

The above-mentioned dosages relate to the compounds of formula 1 in the form of their free bases. If the compounds of formula 1 are administered in the form of their pharmaceutically acceptable acid addition salts, the skilled man can easily calculate the corresponding dosage ranges for the acid addition salts from the dosage ranges specified above, taking into account the molecular weight of the acids used. Particularly preferably, the compounds of formula 1 are administered in the above-mentioned dosage ranges in the form of the enantiomerically pure compounds, particularly preferably in the form of the R-enantiomers thereof.

If the compounds of formula 1 are administered in conjunction with an anticholinergic 2a, the amount of anticholinergic used will fluctuate considerably depending on the choice of active substance.

Without restricting the invention thereto, in the case of tiotropium 2a.1′ amounts of anticholinergic (2a.1′) may be administered such that each single dose contains 0.1-80 μg, preferably 0.5-60 μg, particularly preferably about 1-50 μg of 2a.1′. For example and without restricting the present invention thereto, 2.5 μg, 5 μg, 10 μg, 18 μg, 20 μg, 36 μg or 40 μg 2a.1′ may be administered per single dose. The corresponding amount of salt 2a.1 or of any hydrate or solvate used in each case can easily be calculated by the skilled man, depending on the choice of anion. If for example tiotropium bromide is used as the preferred tiotropium salt 2a.1 according to the invention, the amounts of the active substance 2a.1′ administered per single dose as specified by way of example hereinbefore correspond to the following amounts of 2a.1 administered per single dose: 3 μg, 6 μg, 12 μg, 21.7 μg, 24.1 μg, 43.3 μg and 48.1 μg of 2a.1. In the case of tiotropium 2a.1′ the dosages specified above are preferably administered once or twice a day, while administration once a day is particularly preferred according to the invention.

Without restricting the invention thereto, in the case of the cation 2a.2′ amounts of anticholinergic (2a.2′) may be administered such that each single dose contains 1-500 μg, preferably 5-300 μg, particularly preferably 15-200 μg 2a.2′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.2′ may be administered per single dose. The corresponding amount of salt 2a.2 used in each case or of any hydrate or solvate used can easily be calculated by the skilled man, depending on the choice of anion. In the case of oxitropium 2a.2′ the dosages specified above are preferably administered one to four times a day, while administration two to three times a day is particularly preferred according to the invention.

Without restricting the invention thereto, in the case of the cation 2a.3′ amounts of anticholinergic (2a.3′) may be administered such that each single dose contains 1-500 μg, preferably 5-300 μg, particularly preferably 15-200 μg 2a.3′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.3′ may be administered per single dose. The corresponding amount of salt 2a.3 used in each case or of any hydrate or solvate used can easily be calculated by the skilled man, depending on the choice of anion. In the case of flutropium 2a.3′ the dosages specified above are preferably administered one to four times a day, while administration two to three times a day is particularly preferred according to the invention.

Without restricting the invention thereto, in the case of the cation 2a.4′ amounts of anticholinergic (2a.4′) may be administered such that each single dose contains 1-500 μg, preferably 5-300 μg, particularly preferably 20-200 μg 2a.4′. For example and without restricting the present invention thereto, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.4′ may be administered per single dose. The corresponding amount of salt 2a.4 used in each case or of any hydrate or solvate used can easily be calculated by the skilled man, depending on the choice of anion. In the case of ipratropium 2a.4′ the dosages specified above are preferably administered one to four times a day, while administration two to three times a day, more preferably three times a day, is particularly preferred according to the invention.

Without restricting the invention thereto, in the case of the cation 2a.5′ amounts of anticholinergic (2a.5′) may be administered such that each single dose contains 1-500 μg, preferably 5-300 μg, particularly preferably 15-200 μg. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.5′ may be administered per single dose. The corresponding amount of salt 2a.5 used in each case or of any hydrate or solvate used can easily be calculated by the skilled man, depending on the choice of anion. In the case of glycopyrronium 2a.5′ the dosages specified above are preferably administered one to four times a day, while administration two to three times a day is particularly preferred according to the invention.

Without restricting the invention thereto, in the case of the cation 2a.6′ amounts of anticholinergic (2a.6′) may be administered such that each single dose contains 1000-6500 μg, preferably 2000-6000 μg, particularly preferably 3000-5500 μg, particularly preferably 4000-5000 μg 2a.6′. For example and without restricting the present invention thereto, 3500 μg, 3750 μg, 4000 μg, 4250 μg, 4500 μg, 4750 μg, or 5000 μg of 2a.6′ may be administered per single dose. The corresponding amount of salt 2a.6 used in each case or of any hydrate or solvate used can easily be calculated by the skilled man, depending on the choice of anion. In the case of trospium 2a.6′ the dosages specified above are preferably administered one to four times a day, while administration two to three times a day is particularly preferred according to the invention.

Without restricting the invention thereto, in the case of the cation 2a.7′ amounts of anticholinergic (2a.7′) may be administered such that each single dose contains 50-1000 μg, preferably 100-800 μg, particularly preferably 200-700 μg, particularly preferably 300-600 μg 2a.7′. For example and without restricting the present invention thereto, 300 μg, 350 μg, 400 μg, 450 μg, 500 μg, 550 μg, or 600 μg of 2a.7′ may be administered per single dose. The corresponding amount of salt 2a.7 used in each case or of any hydrate or solvate used can easily be calculated by the skilled man, depending on the choice of anion. In the case of the cation 2a.7′ the dosages specified above are preferably administered one to three times a day, while administration once or twice a day, more preferably once a day, is particularly preferred according to the invention.

Without restricting the invention thereto, in the case of the cations 2a.9′ and 2a.10′, amounts of anticholinergic (2a.9′ or 2a.10′) may be administered such that each single dose contains 1-500 μg, preferably 5-300 μg, particularly preferably 15-200 μg 2a.9′ or 2a.10′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.9′ or 2a.10′ may be administered per single dose. The corresponding amount of salt 2a.9′ or 2a.10′ or of any hydrate or solvate used in each case can easily be calculated by the skilled man, depending on the choice of anion. In the case of the cations 2a.9′ or 2a.10′ the dosages specified above are preferably administered one to three times a day, while administration once or twice a day, more preferably once a day, is particularly preferred according to the invention.

Without restricting the invention thereto, in the case of the cations 2a.11′ to 2a.13′ amounts of anticholinergic (2a.11′, 2a.12′ or 2a.13′) may be administered such that each single dose contains 1-500 μg, preferably 5-300 μg, particularly preferably 10-200 μg 2a.11′, 2a.12′ or 2a.13′. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.11′, 2a.12′ or 2a.13′ may be administered per single dose. The corresponding amount of salt 2a.11, 2a.12 or 2a.13 or of any hydrate or solvate used in each case can easily be calculated by the skilled man, depending on the choice of anion.

In the case of the cations 2a.11, 2a.12 or 2a.13 the dosages specified above are preferably administered one to three times a day, while administration once or twice a day, more preferably once a day, is particularly preferred according to the invention.

If the compounds of formula 1 are administered in combination with a PDE IV-inhibitor 2b, preferably about 1-10000 μg 2b are administered per single dose. Preferably, amounts of 2b are administered such that each single dose contains 10-5000 μg, preferably 50-2500 μg, particularly preferably 100-1000 μg of 2b. For example and without restricting the present invention thereto, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2b may be administered per single dose. In the event that acid addition salts of 2b are used, the corresponding amount of salt used can easily be calculated by the skilled man from the values given hereinbefore, depending on the choice of acid.

If the compounds of formula 1 are administered in combination with a steroid 2c, preferably about 1-10000 μg of 2c are administered per single dose. Preferably, amounts of 2c are administered such that each single dose contains 5-5000 μg, preferably 5-2500 μg, particularly preferably 10-1000 μg of 2c. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2c may be administered per single dose. In the event that salts or derivatives of 2c are used, the corresponding amount of salt/derivative used can easily be calculated by the skilled man from the values given hereinbefore, depending on the choice of salt/derivative.

If the compounds of formula 1 are administered in combination with an LTD4-antagonist 2d, preferably about 0.01-500 mg 2d are administered per single dose. Preferably, amounts of 2d are administered such that each single dose contains 0.1-250 mg, preferably 0.5-100 mg, particularly preferably 1-50 mg of 2d. For example and without restricting the present invention thereto, 1 mg, 2.5 mg, 5 mg, 5.5 mg, 7 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg or 50 mg of 2d may be administered per single dose. In the event that acid addition salts, salts or derivatives of 2d are used, the corresponding amount of salt/derivative used can easily be calculated by the skilled man from the values given hereinbefore, depending on the choice of salt/derivative.

If the compounds of formula 1 are administered in combination with an EGFR-inhibitor 2e, preferably about 100-15000 μg of 2e are administered per single dose. Preferably, amounts of 2e are administered such that each single dose contains 500-10000 μg, preferably 750-8000 μg, particularly preferably 1000-7000 μg of 2e. For example and without restricting the present invention thereto, 1000 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg, 3150 μg, 3200 μg, 3250 μg, 3300 μg, 3350 μg, 3400 μg, 3450 μg, 3500 μg, 3550 μg, 3600 μg, 3650 μg, 3700 μg, 3750 μg, 3800 μg, 3850 μg, 3900 μg, 3950 μg, 4000 μg, 4050 μg, 4100 μg, 4150 μg, 4200 μg, 4250 μg, 4300 μg, 4350 μg, 4400 μg, 4450 μg, 4500 μg, 4550 μg, 4600 μg, 4650 μg, 4700 μg, 4750 μg, 4800 μg, 4850 μg, 4900 μg, 4950 μg, 5000 μg, 5050 μg, 5100 μg, 5150 μg, 5200 μg, 5250 μg, 5300 μg, 5350 μg, 5400 μg, 5450 μg, 5500 μg, 5550 μg, 5600 μg, 5650 μg, 5700 μg, 5750 μg, 5800 μg, 5850 μg, 5900 μg, 5950 μg, 6000 μg, 6050 μg, 6100 μg, 6150 μg, 6200 μg, 6250 μg, 6300 μg, 6350 μg, 6400 μg, 6450 μg, 6500 μg, 6550 μg, 6600 μg, 6650 μg, 6700 μg, 6750 μg, 6800 μg, 6850 μg, 6900 μg, 6950 μg, or 7000 μg of 2e may be administered per single dose. In the event that acid addition salts of 2e are used, the corresponding amount of the salt used can easily be calculated by the skilled man from the values given hereinbefore, depending on the choice of acid.

The two active substance components 1 and 2 may be administered—together or separately—in each case by inhalation or by oral, parenteral or some other route, in known manner, in substantially conventional formulations such as for example plain or coated tablets, pills, granules, aerosols, syrups, emulsions, suspensions, powders and solutions, using inert, non-toxic, pharmaceutically suitable carriers or solvents.

Suitable preparations for administering the compounds of formula 1 and 2 include tablets, capsules, suppositories, solutions, powders, etc. The proportion of pharmaceutically active compound or compounds should be in the range from 0.05 to 90% by weight, preferably 0.1 to 50% by weight of the total composition. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations of active substances according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additional substances such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

Preferably, even when the two components 1 and 2 are administered separately, at least component 1 is administered by inhalation. If component 1 is administered by inhalation, when the two active substances are taken separately, component 2 may also be administered for example by oral or parenteral route using formulations conventional in the art such as plain or coated tablets, pills, granules, aerosols, syrups, emulsions, suspensions, powders and solutions, using inert, non-toxic, pharmaceutically suitable carriers or solvents.

Preferably, however, the medicament combinations according to the invention are administered by inhalation by means of a single preparation containing both active substances 1 and 2 or by means of separate preparations each containing only one of the active substances 1 and 2, suitable for administration by inhalation.

Inhalable preparations include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A) Inhalable Powder Containing the Combinations of Active Substances According to the Invention:

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1 and 2 optionally in conjunction with a physiologically acceptable excipient may be administered, for example, using the inhaler known by the name Turbohaler® or using inhalers as disclosed for example in EP 237507 A. Preferably, the inhalable powders according to the invention which contain physiologically acceptable excipients in addition to 1 and 2 are packed into capsules (to produce so-called inhalettes) which are used in inhalers as described, for example, in WO 94/28958.

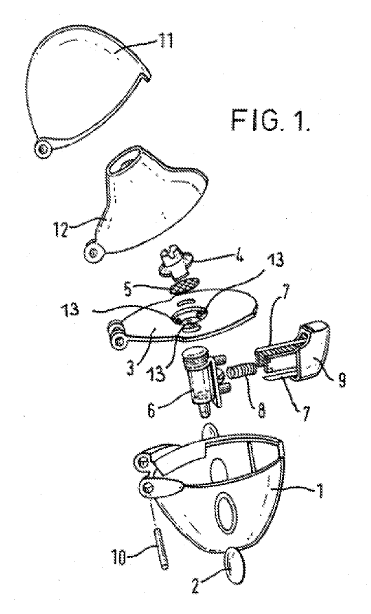

A particularly preferred inhaler for using the pharmaceutical combination according to the invention in inhalettes is shown in FIG. 1. This inhaler (Handihaler®) for inhaling powdered pharmaceutical compositions from capsules is characterised by a housing 1 containing two windows 2, a deck 3 in which there are air inlet ports and which is provided with a screen 5 secured by a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air through-holes 13 for adjusting the flow resistance.

If the inhalable powders according to the invention are to be packaged in capsules, in accordance with the preferred method of administration described above, the capsules should preferably contain from 1 to 30 mg each. According to the invention they contain either together or separately the dosages per single dose specified for 1 and 2 hereinbefore.

B) Propellant Gas-Driven Inhalation Aerosols Containing the Combinations of Active Substances According to the Invention:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

Propellant-free inhalable solutions according to the invention contain for example aqueous or alcoholic, preferably ethanolic solvents, possibly ethanolic solvents in admixture with aqueous solvents. In the case of aqueous/ethanolic solvent mixtures the relative proportion of ethanol to water is not restricted, but the maximum limit is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid, etc. Preferred inorganic acids are hydrochloric acid and sulphuric acid. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

The above-mentioned examples of the active substances 2 are known in the art. The compounds of formula 1 by contrast are not known in the art.

The examples of synthesis described hereinafter serve to illustrate possible methods of synthesising the new compounds of formula 1. However, they are intended only as examples of procedures as an illustration of the invention without restricting the invention to the subject-matter described by way of example.

### Examples

Synthesis of Intermediates

Intermediate 1: tert.butyl(3-amino-3-methyl-butyl)-carbamate: 23.6 g (117 mmol) tert.butyl(1,1-dimethyl-3-oxo-propyl)-carbamate in 700 mL ethanolic ammonia solution are treated in the presence of 3.5 g Raney nickel at ambient temperature under a hydrogen pressure of 3 bar until no more educt can be detected by thin layer chromatography. The catalyst is filtered off and the solvent is eliminated by distillation. Dark green oil.

Yield: 22.7 g (96%); mass spectroscopy: [M+H]⁺=203.

### Intermediate 2: 1-(3-amino-1,1-dimethyl-propyl)-6-methyl-1,3-dihydro-benzimidazol-2-one

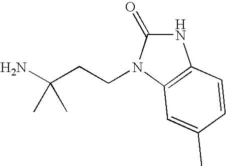

a) tert.butyl[3-methyl-3-(5-methyl-2-nitro-phenylamino)-butyl]-carbamate:

2.0 g (12.9 mmol) 3-fluoro-4-nitro-toluene, 2.6 g (13.0 mmol) tert.butyl(3-amino-3-methyl-butyl)-carbamate and 2.3 g (16.8 mmol) potassium carbonate are stirred overnight at ambient temperature in 20 mL DMF. The solvent is distilled off and the residue is combined with ethyl acetate. The mixture is washed repeatedly with water, dried with sodium sulphate and the solvent is eliminated. 4.8 g yellow oil. Mass spectroscopy: [M+H]⁺=338.

b) tert.butyl[3-(2-amino-5-methyl-phenylamino)-3-methyl-butyl]-carbamate:

4.71 g (14.0 mmol) tert.butyl[3-methyl-3-(5-methyl-2-nitro-phenylamino)-butyl]carbamate are dissolved in 110 mL methanol and hydrogenated in the presence of 340 mg palladium on charcoal (10%) at ambient temperature. Then the catalyst is separated off and the solvent is distilled off. Brown solid. Yield: 3.72 g (87%); mass spectroscopy: [M+H]⁺=308.

c) tert.butyl[3-methyl-3-(6-methyl-2-oxo-2,3-dihydro-benzimidazol-1-yl)-butyl]-carbamate:

1.76 g (5.7 mmol) tert.butyl[3-(2-amino-5-methyl-phenylamino)-3-methyl-butyl]carbamate are dissolved in 35 mL THF, combined with 2.1 g (12.7 mmol) 1,1′-carbonyldi-(1,2,4-triazole) and stirred overnight. The solvent is distilled off and the residue is dissolved in ethyl acetate. The solution is washed successively with potassium hydrogen sulphate solution and sodium chloride solution and dried with sodium sulphate. The residue is chromatographed (silica gel; dichloromethane with 0-16% methanol:ammonia=9:1) and the crude product thus obtained is stirred with diethyl ether. Light yellow solid. Yield: 1.12 g (59%); mass spectroscopy: [M+H]⁺=334.

d) 1-(3-amino-1,1-dimethyl-propyl)-6-methyl-1,3-dihydro-benzimidazol-2-one: a solution of 1.50 g (4.5 mmol) tert.butyl[3-methyl-3-(6-methyl-2-oxo-2,3-dihydro-benzimidazol-1-yl)-butyl]-carbamate in 100 mL dioxane is combined with 10 mL 4 molar hydrochloric acid in dioxane and then heated for 90 minutes to 90° C., during which time a white precipitate settles out. After cooling to ambient temperature the solvent is distilled off and the residue is stirred in diethyl ether. White solid.

Yield: 1.04 g (86%; hydrochloride); mass spectroscopy: [M+H]⁺=234.

### Intermediate 3:1-(3-amino-3-methyl-butyl)-5-trifluoromethyl-1,3-dihydro-benzimidazol-2-one

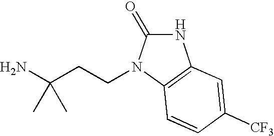

a) tert.butyl[3-methyl-3-(2-nitro-4-trifluoromethyl-phenylamino)-butyl]-carbamate: This is prepared analogously to Method 2a) from a total of 3.25 g (15.5 mmol) 1-fluoro-2-nitro-4-trifluoromethyl-benzene and 2.74 g (13.5 mmol) tert.butyl(3-amino-3-methyl-butyl)-carbamate. 6.1 g yellow oil. Mass spectroscopy: [M+H]⁺=392.

b) tert.butyl[3-(2-amino-4-trifluoromethyl-phenylamino)-1,1-dimethyl-propyl]-carbamate:

6.10 g (15.6 mmol) tert.butyl[3-methyl-3-(2-nitro-4-trifluoromethyl-phenylamino)-butyl]-carbamate are hydrogenated analogously to Method 2b).

Yield: 5.05 g (90%); mass spectroscopy: [M+H]⁺=362.

c) tert.butyl[1,1-dimethyl-3-(2-oxo-5-trifluoromethyl-2,3-dihydro-benzimidazol-1-yl)-propyl]-carbamate: 5.00 g (13.8 mmol) tert.butyl[3-(2-amino-4-trifluoromethyl-phenylamino)-1,1 -dimethyl-propyl]-carbamate and 6.73 g (41.5 mmol) 1,1′-carbonyldiimidazole are reacted and worked up analogously to Method 2c). White solid. Yield: 4.18 g (78%); mass spectroscopy: [M−H]⁺=386.

d) 1-(3-amino-3-methyl-butyl)-5-trifluoromethyl-1,3-dihydro-benzimidazol-2-one: Prepared analogously to Method 2d) from 2.89 g (7.5 mmol) tert.butyl[1,1-dimethyl-3-(2-oxo-5-trifluoromethyl-2,3-dihydro-benzimidazol-1-yl)-propyl]-carbamate.

Yield: 1.60 g (66%); mass spectroscopy: [M+H]⁺=288.

### Intermediate 4: 3-(3-amino-3-methyl-butyl)-3H-benzoxazol-2-one

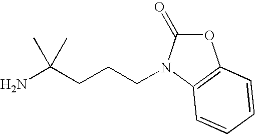

a) 1-iodo-4-methyl-nitro-pentane: A solution of 44.7 mL (352 mmol) chlorotrimethylsilane and 50 mL acetonitrile is added dropwise to 26.0 g (177 mmol) 1-methyl-4-nitro-pentan-1-ol and 52.8 g (352 mmol) sodium iodide in 350 mL acetonitrile. Then the mixture is heated to 50° C. for 4 hours, then the solvent is distilled off and the residue is combined with 500 mL diethyl ether. It is washed successively with water, sodium thiosulphate solution and sodium chloride solution. The organic phase is dried with sodium sulphate and evaporated down. 34.2 g red oil.

b) 3-(3-methyl-3-nitro-butyl)-3H-benzoxazol-2-one: 1.70 g (42.5 mmol) sodium hydride (60%) are added batchwise to a solution of 4.50 g (33.3 mmol) benzoxazol-2-one in 50 mL DMF, while the temperature is kept below 0° C. by cooling. After one hour's stirring a solution of 9.61 g (37.4 mmol) 1-iodo-4-methyl-4-nitro-pentane in 20 mL DMF is added dropwise such that the temperature does not rise above 5° C. The mixture is left overnight at ambient temperature with stirring and the solvent is distilled off. The residue is taken up in ethyl acetate and washed successively with water and sodium chloride solution, dried with sodium sulphate and evaporated down. 11.0 g oil are obtained. Mass spectroscopy: [M+H]⁺=265.

c) 3-(3-amino-3-methyl-butyl)-3H-benzoxazol-2-one: 11.0 g 3-(3-methyl-3-nitro-butyl)-3H-benzoxazol-2-one from the reaction described above are dissolved in 130 mL ethanol and hydrogenated with Raney nickel as catalyst at 5 bar over 20 hours. The catalyst is filtered off and the filtrate is freed from the solvent. 10% ethanolic hydrochloric acid is added, the solvent is distilled off and the residue is stirred in an acetone/diethyl ether mixture. White solid. Yield: 6.0 g (77% over 2 steps, hydrochloride); melting range=145-147° C.

### Intermediate 5: 3-(3-amino-3-methyl-butyl)-3H-benzoxazol-2-one

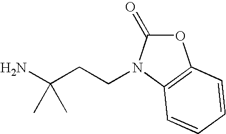

a) tert.butyl[1,1-dimethyl-3-(2-oxo-benzoxazol-3-yl)-propyl]-carbamate:

4.0 g (29.6 mmol) benzoxazol-2-one are dissolved in 40 mL DMPU and cooled with an ice bath. 897 mg (95%; 35.5 mmol) sodium hydride are added batchwise to this solution under protective gas. The reaction mixture is heated to ambient temperature and then stirred for another hour. 9.85 g (44.4 mmol) tert.butyl(3-amino-1,1-dimethyl-propyl)-carbamate and 1.97 g (5.3 mmol) tetrabutylammonium iodide are added and the mixture is stirred overnight. The reaction is stopped by the careful addition of sodium hydrogen carbonate solution. Ethyl acetate is added, the aqueous phase is separated off and extracted repeatedly with ethyl acetate. The combined organic phases are washed with sodium chloride solution, dried with sodium sulphate and evaporated down. Purification of the residue by column chromatography (silica gel; petroleumether/ethyl acetate=7:3) yields the desired product in the form of an oil.

Yield 4.1 g (43%); mass spectroscopy: [M+H]⁺=321.

b) 3-(3-amino-3-methyl-butyl)-3H-benzoxazol-2-one:

18 mL trifluoroacetic acid are added dropwise at ambient temperature to a solution of 4.0 g (12.5 mmol) tert.butyl[1,1-dimethyl-3-(2-oxo-benzoxazol-3-yl)-propyl]-carbamate in 110 mL dichloromethane. The mixture is left overnight with stirring and then the solvent is distilled off. The oil remaining is stirred into diethyl ether, during which time a solid is precipitated which is filtered off. After renewed stirring with diethyl ether and filtration a beige solid is obtained.

Yield: 3.63 g (65%; trifluoroacetate); mass spectroscopy: [M+H]⁺=221.

### Intermediate 6: 5-benzyloxy-7-(2-ethoxy-2-hydroxy-acetyl)-3H-benzoxazol-2-one

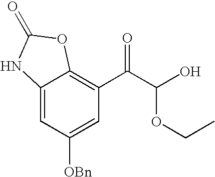

a) 1-(5-benzyloxy-2-hydroxy-3-nitro-phenyl)-ethanone:

18 mL fuming nitric acid are added dropwise to a solution of 81.5 g (0.34 mol) 1-(5-benzyloxy-2-hydroxy-phenyl)-ethanone (known from U.S. Pat. No. 4,460,581) in 700 mL acetic acid, while being cooled with the ice bath, in such a way that the temperature does not rise above 20° C. Then the reaction mixture is stirred for two hours at ambient temperature, poured onto ice water and filtered. The product is recrystallised from isopropanol, suction filtered and washed with isopropanol and diisopropylether.

Yield: 69.6 g (72%); mass spectroscopy [M+H]⁺=288.

b) 1-(3-amino-5-benzyloxy-2-hydroxy-phenyl)-ethanone:

69.5 g (242 mmol) 1-(5-benzyloxy-2-hydroxy-3-nitro-phenyl)-ethanone are dissolved in 1.4 L methanol and hydrogenated in the presence of 14 g rhodium on charcoal (10%) as catalyst at 3 bar and ambient temperature. Then the catalyst is filtered off and the filtrate is evaporated down. The residue is further reacted without any additional purification.

Yield: 60.0 g (96%), R_{f}value=0.45 (dichloromethane on silica gel).

c) 7-acetyl-5-benzyloxy-3H-benzoxazol-2-one: 52 g (0.53 mol) phosgene are piped into a solution of 121 g (0.47 mol) 1-(3-amino-5-benzyloxy-2-hydroxy-phenyl)-ethanone in 800 mL pyridine at 20 to 40° C. The reaction mixture is heated to 50° C. for 2 hours, then poured onto ice and acidified with conc. hydrochloric acid. A red-brown solid is isolated, which is repeatedly recrystallised from ethanol with the addition of activated charcoal.

Yield: 67.5 g (50.6%); Melting range: 163-166° C.

d) 5-benzyloxy-7-(2-ethoxy-2-hydroxy-acetyl)-3H-benzoxazol-2-one:

20 g (71 mmol) 7-acetyl-5-benzyloxy-3H-benzoxazol-2-one and 8 g (72 mmol) selenium dioxide are stirred at reflux temperature in the presence of activated charcoal in 100 mL dioxane and 3.1 mL water for 8 hours. The solid is filtered off, the solvent is distilled off and the residue is combined with 50 mL ethanol. The mixture is refluxed for 15 minutes and then filtered through activated charcoal. The solid precipitated on cooling is suction filtered after 3 hours and washed with ethanol and diethyl ether.

Yield: 7 g (29%); Melting range: 140-143° C.

### Intermediate 7: 6-benzyloxy-8-(2-ethoxy-2-hydroxy-acetyl)-2,2-dimethyl-4H-benzo[1,4]oxazin-3-one

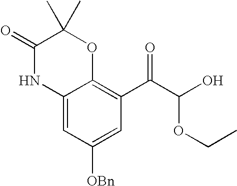

a) N-(3-acetyl-5-benzyloxy-2-hydroxy-phenyl)-2-bromo-2-methyl-propionamide:

4.64 g (25 mmol) 2-bromo-2-methyl-propionyl chloride are added dropwise at 5 to 20° C. to a solution of 5.15 g (20 mmol) 1-(3-amino-5-benzyloxy-2-hydroxy-phenyl)-ethanone in 20 mL pyridine. After the addition has ended the mixture is stirred for 15 minutes, combined with ice water and 100 mL ethyl acetate and acidified with conc. hydrochloric acid. The organic phase is separated off, washed with water and dried with sodium sulphate. After the solvent has been distilled off the residue is recrystallised from a diethyl ether/petroleumether mixture. Yield: 6.8 g (84%); Melting range: 88-90° C.

b) 8-acetyl-6-benzyloxy-2,2-dimethyl-4H-benzo[1,4]oxazin-3-one:

6.60 g (16.2 mmol) N-(3-acetyl-5-benzyloxy-2-hydroxy-phenyl)-2-bromo-2-methyl-propionamide and 2.76 g (20 mmol) potassium carbonate are stirred for 1 hour in 70 mL acetonitrile at reflux temperature. The solid is suction filtered, the filtrate is evaporated down and the residue is combined with 30 mL ethyl acetate. After renewed filtration and after the solvent has been distilled off the crude product is crystallised from a little methanol.

Yield: 1.00 g (19%); mass spectroscopy [M+H]⁺=326; Melting range: 148-150° C.

c) 6-benzyloxy-8-(2-ethoxy-2-hydroxy-acetyl)-2,2-dimethyl-4H-benzo[1,4]oxazin-3-one:

Prepared analogously to the method described for Intermediate 6d from 8-acetyl-6-benzyloxy-2,2-dimethyl-4H-benzo[1,4]oxazin-3-one.

### Intermediate 8: 7-benzyloxy-5-oxiranyl-1H-quinolin-2-one

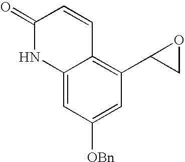

a) 2-acetyl-4-benzyloxy-6-nitro-phenyl trifluoromethanesulphonate:

92.7 mL (660 mmol) triethylamine are added to 90 g (313 mmol) 1-(5-benzyloxy-2-hydroxy-3-nitro-phenyl)-ethanone in 940 mL dichloromethane at −10° C. Then a solution of 65 mL (394 mmol) trifluoromethanesulphonic anhydride and 40 mL dichloromethane is slowly added dropwise. After 15 minutes stirring at −5° C. the reaction is stopped by the careful addition of 400 mL ammonium chloride solution and 400 mL sodium hydrogen carbonate solution. The organic phase is separated off, dried with sodium sulphate and evaporated down. The residue is dissolved in 150 mL diethyl ether and then precipitated by the addition of 800 mL hexane. The solid is filtered off, suspended in a diethyl ether/hexane mixture and suction filtered again.

Yield: 118 g (90%); mass spectroscopy: [M+H]⁺=420.

b) methyl 3-(2-acetyl-4-benzyloxy-6-nitro-phenyl)-acrylate: 5.88 g (6.42 mmol) tris-(dibenzylideneacetone)-dipalladium, 3.50 g (12.01 mmol) tri-tert-butylphosphonium tetrafluoroborate, 81.2 mL (371 mmol) dicyclohexylmethylamine, 105.8 g (286 mmol) tetrabutylammonium iodide and 32.6 mL (362 mmol) methyl acrylate are added to a solution of 100 g (238 mmol) 2-acetyl-4-benzyloxy-6-nitro-phenyl trifluoromethanesulphonate in 360 mL dioxane. The reaction mixture is stirred for 2 hours at 80° C. under a nitrogen atmosphere in the presence of 100 g molecular sieve 4A and then combined with 2 L diethyl ether and 500 g silica gel. After 10 minutes the silica gel is suction filtered, while washing repeatedly with diethyl ether. The combined organic phases are washed successively with 1 N hydrochloric acid, sodium carbonate solution and sodium chloride solution. The solvent is distilled off, the residue is crystallised from ethanol and the solid is filtered off and washed with ethanol.

Yield: 32.2 g (38%); mass spectroscopy: [M+H]⁺=356.

c) 5-acetyl-7-benzyloxy-3,4-dihydro-1H-quinolin-2-one: 5.0 g (14.07 mmol) methyl 3-(2-acetyl-4-benzyloxy-6-nitro-phenyl)-acrylate are combined with 100 mL ethanol and hydrogenated with Raney nickel as catalyst at 4 bar. The catalyst is separated off and the filtrate is acidified with 15 mL of 2 N hydrochloric acid. The product that crystallises out is suction filtered and dried. Yield: 1.0 g (24%); mass spectroscopy: [M+H]⁺=296.

d) 5-acetyl-7-benzyloxy-1H-quinolin-2-one: 13.0 g (44 mmol) 5-acetyl-7-benzyloxy-3,4-dihydro-1H-quinolin-2-one are suspended in 130 mL dioxane and combined with 15.0 g (66 mmol) 2,3-dichloro-5,6-dicyanobenzoquinone. The mixture is refluxed for 30 minutes, cooled to ambient temperature and stirred for another 2 hours. The solid is filtered off, washed with dioxane and dissolved in 600 mL dichloromethane/methanol (9:1). The solution is washed with sodium hydrogen carbonate solution, dried with sodium sulphate and evaporated down. Then the residue is suspended in methanol, filtered and dried.

Yield: 8.3 g (64%); mass spectroscopy: [M+H]⁺=294.

e) 7-benzyloxy-5-(2-chloro-acetyl)-1H-quinolin-2-one:

7.0 g (23.9 mmol) 5-acetyl-7-benzyloxy-1H-quinolin-2-one and 19.0 g (54.6 mmol) benzyltrimethylammonium dichloriodate are stirred in 43 mL acetic acid, 7 mL water and 147 mL dichloroethane at 65° C. After 4.5 hours the reaction is stopped by the addition of 400 mL sodium carbonate solution and 50 mL of 5% sodium sulphite solution. The insoluble constituents are suction filtered, washed with water and dried.

Yield: 6.0 g (77%); mass spectroscopy: [M+H]⁺=328.

f) 7-benzyloxy-5-oxiranyl-1H-quinolin-2-one:

6.0 g (18.3 mmol) 7-benzyloxy-5-(2-chloro-acetyl)-1 H-quinolin-2-one are placed in 150 mL tetrahydrofuran and at 0 to 5° C. combined with 434 mg (19.9 mmol) lithium borohydride. The mixture is stirred for 30 minutes, then 43 mL of a 2.5 molar sodium hydroxide solution are added and stirring is continued for a further 4 hours with heating to ambient temperature. The mixture is combined with sodium chloride solution, filtered and repeatedly extracted with ethyl acetate/tetrahydrofuran (1:1). The solid filtered off and the organic phases are combined and freed from the solvent. The residue is suspended in methanol, suction filtered and dried. Yield 4.8 g (89%); mass spectroscopy: [M+H]⁺=294.

### Intermediate 9: 1-(3-amino-3-methyl-butyl)-4-methoxy-1,3-dihydro-benzimidazol-2-one

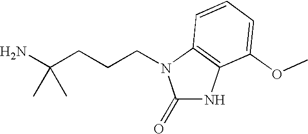

a) 4-methyl-4-nitro-pentan-1-ol: 50 g (0.285 mol) methyl 4-methyl-4-nitro-pentanoate are dissolved in a 6:4 mixture von of THF/ethanol (1000 mL). The solution is cooled to −10° C. and combined with 24.2 g (0.571 mol) lithium chloride. Then 21.6 g (0.571 mol) lithium borohydride are added batchwise. The mixture is stirred for 30 minutes at −10° C. and then heated overnight to ambient temperature. The reaction mixture is stirred for 6 hours at 60° C. and overnight at ambient temperature. It is combined with water and adjusted to pH 6 with dilute hydrochloric acid. The solvent is distilled off and the residue with is combined with water. The mixture is extracted with dichloromethane, the combined organic phases are washed with water and ammonium chloride solution and dried with sodium sulphate. After elimination of the solvent the product is obtained as a yellow oil.

Yield: 40.0 g (95%); mass spectroscopy: [M+H]⁺=148.

b) 1-iodo-4-methyl-4-nitro-pentane: 70 mL (0.544 mol) trimethylchlorosilane are added dropwise at ambient temperature to 40 g (0.272 mol) 4-methyl-4-nitro-pentan-1-ol and 81.5 g (0.544 mol) sodium iodide in 350 mL acetonitrile. The reaction mixture is filtered, evaporated down and combined with diethyl ether. The organic phase is washed with sodium bisulphite solution and water, dried and freed from the solvent. Yellow oil.

Yield: 56.0 g (80%); mass spectroscopy: [M−NO₂]⁺=211.

c) 2-methoxy-6-nitro-phenylamine: 85% potassium hydroxide solution (11.7 g, 0.179 mol) is added to a solution of 25 g (0.162 mol) 2-amino-3-nitro-phenol in 200 mL DMF. Then 11.1 mL (0.178 mol) iodomethane are added dropwise and the mixture is stirred overnight at ambient temperature. The reaction mixture is poured onto ice and stirred for one hour. The precipitated product is filtered off, washed with water and dried.

Yield: 23.8 g (87%); mass spectroscopy: [M+H]⁺=169.

d) ethyl(2-methoxy-6-nitro-phenyl)-carbamate:

At reflux temperature 17.1 mL (0.141 mol) trichloromethyl chloroformate are added dropwise to a solution of 23.8 g (0.142 mol) 2-methoxy-6-nitro-phenylamine in 300 mL THF and then stirred for 4 hours at this temperature. The solvent is distilled off and the residue is stirred with isopropanol, whereupon a yellow solid is precipitated.

Yield: 25.0 g (73%); mass spectroscopy: [M+H]⁺=241.

e) ethyl(2-amino-6-methoxy-phenyl)-carbamate:

25.0 g (0.104 mol) ethyl(2-methoxy-6-nitro-phenyl)-carbamate are dissolved in 400 mL methanol. 116.4 g (0.516 mol) SnCl₂2H₂O are added and the mixture is refluxed for 3 hours. The reaction mixture is evaporated down, combined with sodium carbonate solution and filtered. The aqueous phase is again extracted with dichloromethane and the combined organic phases are washed with sodium chloride solution, dried and evaporated down. The residue that crystallises out on standing is stirred with isopropanol.

Yield: 13.0 g (59%); mass spectroscopy: [M+H]⁺=211.

f) ethyl 7-methoxy-2-oxo-2,3-dihydro-benzimidazole-1-carboxylate: While cooling with ice 13.0 g (0.062 mol) ethyl(2-amino-6-methoxy-phenyl)-carbamate and 10.3 mL (0.074 mol) triethylamine in 100 mL dichloromethane are added to a solution of 8.20 mL (0.068 mol) trichloromethyl chloroformate in 50 mL dichloromethane. After 4 hours stirring at ambient temperature the reaction mixture is poured onto ice and extracted with dichloromethane. The combined organic phases are washed with water, dried and freed from the solvent. The residue is stirred in diethyl ether.

Yield: 9.0 g (62%); mass spectroscopy: [M+H]⁺=237.

g) 4-methoxy-1-(3-methyl-3-nitro-butyl)-1,3-dihydro-benzimidazol-2-one: 4.0 g (17 mmol) ethyl 7-methoxy-2-oxo-2,3-dihydro-benzimidazole-1-carboxylate in DMF are combined with 85% potassium hydroxide solution (3.3 g, 51 mmol) while being cooled with the ice bath. After 30 minutes a solution of 5.2 g (21 mmol) 1-iodo-4-methyl-4-nitro-pentane in DMF is added and the mixture is stirred overnight at ambient temperature. The reaction mixture is diluted with water and extracted with ethyl acetate. The combined organic phases are washed with water, dried and freed from the solvent. The oil remaining is purified by chromatography on a silica gel column (cyclohexane/ethyl acetate gradient).

Yield: 0.5 g (8%); mass spectroscopy: [M+H]⁺=366.

h) 1-(3-amino-3-methyl-butyl)-4-methoxy-1,3-dihydro-benzimidazol-2-one:

1.4 g (4.8 mmol) 4-methoxy-1-(3-methyl-3-nitro-butyl)-1,3-dihydro-benzimidazol-2-one are dissolved in methanol and hydrogenated in the presence of Raney nickel at 3 bar. The catalyst is separated off, the solvent is distilled off and the residue is dissolved in ethanolic hydrochloric acid. The solvents are removed by distillation and the solid remaining is stirred with isopropanol.

Yield: 0.6 g (42%, hydrochloride); mass spectroscopy: [M+H]⁺=300.

### Intermediate 10: 1-(3-amino-3-methyl-butyl)-5-methoxy-3-methyl-1,3-dihydro-benzimidazol-2-one

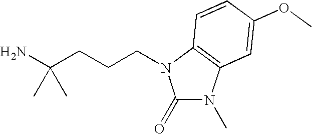

a) (5-methoxy-2-nitro-phenyl)-methyl-amine:

83.5 mL (167.0 mmol) of a 2 molar solution of methylamine in THF are added dropwise to 14.3 g (83.56 mmol) 3-fluoro-4-nitro-anisol and 12.71 g (92.02 mmol) potassium carbonate in 200 mL dichloromethane. The mixture is stirred overnight and then combined with water. The organic phase is washed successively with water and ammonium chloride solution, dried and evaporated down. The yellow solid that remains is stirred with hexane. Yield: 12.7 g (84%); mass spectroscopy: [M+H]⁺=183.

b) 4-methoxy-N-2-methyl-benzene-1,2-diamine: 12.5 g (68.6 mmol) (5-methoxy-2-nitro-phenyl)-methyl-amine and 77.39 g (343.0 mmol) SnCl₂ 2H₂O in 200 mL ethanol are heated to reflux temperature for 6 hours. The reaction mixture is washed with sodium carbonate solution, filtered and evaporated down. The residue is combined with water and extracted with ethyl acetate. The combined organic phases are washed with water, dried and freed from the solvent. Oil. Yield: 8.0 g (77%); mass spectroscopy: [M+H]⁺=153.

c) 5-methoxy-1-methyl-1,3-dihydro-benzimidazol-2-one: 8.0 g (52.56 mmol) 4-methoxy-N-2-methyl-benzene-1,2-diamine and 8.7 mL (63.00 mmol) triethylamine are dissolved in 100 mL dichloromethane and added dropwise to 7 mL (58.00 mmol) trichloromethyl chloroformate in 50 mL dichloromethane. The reaction mixture is stirred overnight at ambient temperature, the poured into ice water and extracted with dichloromethane. The combined organic phases are washed with water, dried and evaporated down. The solid that remains is stirred with diethyl ether.

Yield: 4.2 g (45%); mass spectroscopy: [M+H]⁺=179.

d) 5-methoxy-3-methyl-1-(3-methyl-3-nitro-butyl)-1,3-dihydro-benzimidazol-2-one:

1.1 g (28 mmol) 60% sodium hydride are added to 2.5 g (14 mmol) 5-methoxy-1-methyl-1,3-dihydro-benzimidazol-2-one in 30 mL DMF while being cooled with the ice bath. After 30 minutes a solution of 1-iodo-4-methyl-4-nitro-pentane in 20 mL DMF is piped in and the mixture is stirred overnight. It is diluted with water and extracted with ethyl acetate. The combined organic phases are washed with water, dried and evaporated down. The solid remaining is diluted with diethyl ether.

Yield: 2.7 g (63%); mass spectroscopy: [M+H]⁺=308.

e) 1-(3-amino-3-methyl-butyl)-5-methoxy-3-methyl-1,3-dihydro-benzimidazol-2-one: 2.7 g (8.7 mmol) 5-methoxy-3-methyl-1-(3-methyl-3-nitro-butyl)-1,3-dihydro-benzimidazol-2-one and 9.93 g (44.0 mmol) SnCl₂ 2H₂O in 200 mL ethanol are refluxed for 3 hours. The reaction mixture is evaporated down, combined with sodium carbonate solution and filtered. The filtrate is extracted with ethyl acetate and the combined organic phases are washed with water, dried and freed from the solvent. The residue is dissolved in ethanol and the solution is combined with ethereal hydrochloric acid. After the solvent has been distilled off the solid remaining is stirred with diisopropylether.

Yield: 0.7 g (29%); mass spectroscopy: [M+H]⁺=278.

### Intermediate 11: 3-(4-amino-4-methyl-pentyl)-5-fluoro-1-methyl-1,3-dihydro-benzimidazol-2-one

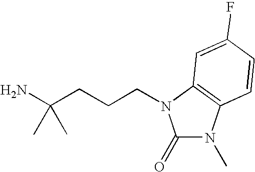

a) (4-fluoro-2-nitro-phenyl)-methyl-amine:

157 ml (314 mmol) of a 2 molar solution of methylamine in THF are added dropwise to 25 g (157 mmol) 2.4-difluoro-nitrobenzene and 23.9 g (173 mmol) potassium carbonate in 300 mL dichloromethane while cooling. The mixture is stirred overnight at ambient temperature and then combined with water. The organic phase is washed with water, dried and evaporated down. The residue is stirred with diethyl ether.

Yield: 18 g (69%); mass spectroscopy [M+H]⁺=171.

b) 4-fluoro-N-1-methyl-benzene-1,2-diamine: 22 g (0.12 mol) (4-fluoro-2-nitro-phenyl)-methyl-amine in 250 mL ethanol are hydrogenated with palladium on charcoal as catalyst at 4 bar hydrogen pressure. The catalyst is separated off and the solvent is distilled off. The oil remaining is purified by chromatography (silica gel, hexane/ethyl acetate gradient). The product is obtained in the form of an oil.

Yield: 9 g (50%); mass spectroscopy [M+H]⁺=141.

c) 5-fluoro-1-methyl-1,3-dihydro-benzimidazol-2-one:

13.0 g (92.1 mmol) 4-fluoro-N-1-methyl-benzene-1,2-diamine are reacted with trichloromethyl chloroformate analogously to the method described for Intermediate 10c. After stirring in diethyl ether the product is isolated as a solid.

Yield: 6.0 g (39%); mass spectroscopy: [M+H]⁺=167.

d) 5-fluoro-1-methyl-3-(4-methyl-4-nitro-pentyl)-1,3-dihydro-benzimidazol-2-one:

First of all 0.624 g (13.9 mmol) 60% sodium hydride and then while cooling 4.6 g (17.8 mmol) 1-iodo-4-methyl-4-nitro-pentane in 10 mL DMF are added to a solution of 2.1 g (12.6 mmol) 5-fluoro-1-methyl-1,3-dihydro-benzimidazol-2-one in DMF. The reaction mixture is stirred overnight at ambient temperature, then poured onto water and extracted with diethyl ether. The organic phases are evaporated down and the residue is recrystallised from isopropylether. Yield: 1.8 g (48%); mass spectroscopy [M+H]⁺=296.

e) 3-(4-amino-4-methyl-pentyl)-5-fluoro-1-methyl-1,3-dihydro-benzimidazol-2-one:

1.8 g (6.09 mmol) 5-fluoro-1-methyl-3-(4-methyl-4-nitro-pentyl)-1,3-dihydro-benzimidazol-2-one in 50 mL methanol are hydrogenated with Raney nickel as catalyst at 3 bar hydrogen pressure. The catalyst is separated off and the solvent is distilled off. In order to prepare the hydrochloride the residue is combined with ethanol and hydrochloric acid in diethyl ether. Yield: 1.5 g (83%, hydrochloride); Melting range=225-228° C.; mass spectroscopy [M+H]⁺=303.

### Intermediate 12: 3-(4-amino-4-methyl-pentyl)-4-fluoro-1-methyl-1,3-dihydro-benzimidazol-2-one

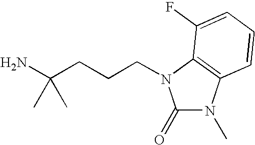

a) (3-fluoro-2-nitro-phenyl)-methyl-amine:

reaction of 2.0 g (2.6 mmol) 2,6-difluoro-nitrobenzene with a 2 molar solution of methylamine in THF analogously to the process for preparing Intermediate 10a. Red solid. Yield: 1.8 g (86%); mass spectroscopy: [M+H]⁺=171.

b) 3-fluoro-N-1-methyl-benzene-1,2-diamine:

reduction of 8.0 g (47.0 mmol) (3-fluoro-2-nitro-phenyl)-methyl-amine with SnCl₂×2H₂O according to the method described for Intermediate 10b. Red oil.

Yield: 4.5 g (68%); mass spectroscopy: [M+H]⁺=141.

c) 4-fluoro-1-methyl-1,3-dihydro-benzimidazol-2-one: Prepared from 4.5 g (32.1 mmol) 3-fluoro-N-1-methyl-benzene-1,2-diamine analogously to the method described for Intermediate 10c. Brown solid. Yield: 1.4 g (26%); mass spectroscopy: [M+H]⁺=167.

d) 4-fluoro-1-methyl-3-(4-methyl-4-nitro-pentyl)-1,3-dihydro-benzimidazol-2-one:

Prepared from 1.4 g (8.42 mmol) 4-fluoro-1-methyl-1,3-dihydro-benzimidazol-2-one analogously to the method described for Intermediate 10d. Yellow oil.

Yield: 1.7 g (68%); mass spectroscopy: [M+H]⁺=296.

e) 3-(4-amino-4-methyl-pentyl)-4-fluoro-1-methyl-1,3-dihydro-benzimidazol-2-one:

A solution of 2 g (6.7 mmol) 4-fluoro-1-methyl-3-(4-methyl-4-nitro-pentyl)-1,3-dihydro-benzimidazol-2-one in methanol is hydrogenated in the presence of Raney nickel at 3 bar hydrogen pressure. After separation of the catalyst hydrochloric acid in diethyl ether is added. The hydrochloride precipitated is filtered off and dried. Yield: 1.5 g (83%, hydrochloride); Melting range=230-232° C.; mass spectroscopy: [M+H]⁺=303.

Synthesis of End Compounds

General Method 1: 1 mmol of glyoxalaldehyde or -acetal and 1 mmol amine are stirred for 30 minutes in 5 mL tetrahydrofuran at 50° C. The mixture is cooled to 0° C. and under an argon atmosphere 1.5 mL of a 2 molar solution of lithium borohydride in tetrahydrofuran is added dropwise. The mixture is stirred for 30 min at 0° C., combined with 10 mL dichloromethane and 3 mL water, stirred for another hour at ambient temperature and then filtered through kieselguhr, while eluting with dichloromethane. The eluate is freed from the solvent and the residue is purified by chromatography, if necessary. The benzylether thus obtained is dissolved in methanol and hydrogenated with palladium on charcoal (10%) as catalyst at 2.5 bar and ambient temperature. Then the catalyst is separated off and the crude product is purified by chromatography (reverse phase, acetonitrile/water gradient with 0.1% trifluoroacetic acid) or crystallised in acetonitrile.

General Method 2: 1 mmol of glyoxalaldehyde or -acetal and 1 mmol amine are suspended in 5 mL ethanol and heated to 70° C. The resulting solution is stirred for one hour at 70° C. and then cooled to ambient temperature. After the addition of 113 mg (3 mmol) sodium borohydride the mixture is stirred for 3 hours at ambient temperature, combined with 0.7 mL saturated potassium carbonate solution and stirred for another 30 minutes. It is filtered through aluminium oxide (basic), repeatedly washed with dichloromethane/methanol 15:1, evaporated down and chromatographed (silica gel; dichloromethane with 0-10% methanol:ammonia=9:1). The benzyl compound thus obtained is dissolved in 10 mL methanol and hydrogenated with palladium on charcoal as catalyst at 1 bar hydrogen pressure. Then the catalyst is filtered off and the filtrate is evaporated down.

### Example 1.1

### 8-{2-[1,1-dimethyl-3-(6-methyl-2-oxo-2,3-dihydro-benzimidazol-1-yl)-propylamino]-1-hydroxy-ethyl}-6-hydroxy-4H-benzo[1,4]oxazin-3-one

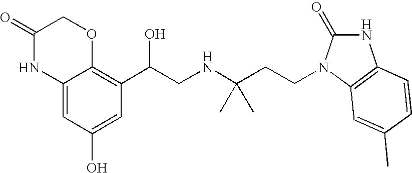

The compound is prepared according to General Method 1 from 357 mg (1 mmol) 6-benzyloxy-8-(2-ethoxy-1,2-dihydroxy-ethyl)-4H-benzo[1,4]oxazin-3-one and 233 mg (1 mmol) 1-(3-amino-3-methyl-butyl)-6-methyl-1,3-dihydro-benzimidazol-2-one.

Yield: 170 mg (31%, trifluoroacetate); mass spectroscopy: [M+H]⁺=441.

### Example 1.2

### 8-{2-[1,1-dimethyl-3-(2-oxo-5-trifluoromethyl-2,3-dihydro-benzimidazol-1-yl)-propylamino]-1-hydroxy-ethyl}-6-hydroxy-4H-benzo[1,4]oxazin-3-one

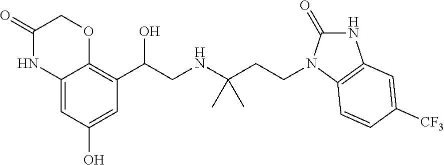

Prepared according to General Method 1 from 357 mg (1 mmol) 6-benzyloxy-8-(2-ethoxy-1,2-dihydroxy-ethyl)-4H-benzo[1,4]oxazin-3-one and 287 mg (1 mmol) 1-(3-amino-3-methyl-butyl)-5-trifluoromethyl-1,3-dihydro-benzimidazol-2-one.

Yield: 76 mg (13%, trifluoroacetate); mass spectroscopy: [M+H]⁺=495.

### Example 1.3

### 8-{2-[1,1-dimethyl-4-(2-oxo-benzoxazol-3-yl)-butylamino]-1-hydroxy-ethyl}-6-hydroxy-4H-benzo[1,4]oxazin-3-one

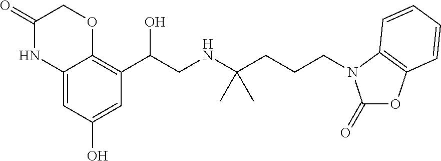

357 mg (1 mmol) 6-benzyloxy-8-(2-ethoxy-1,2-dihydroxy-ethyl)-4H-benzo[1,4]oxazin-3-one and 287 mg (1 mmol) 3-(4-amino-4-methyl-pentyl)-3H-benzoxazol-2-one are reacted according to General Method 1. After hydrogenolytic cleaving of the benzyl protecting group an oil is isolated from which the product is obtained by stirring in an acetone/diethyl ether mixture. Yield: 161 mg (29%, trifluoroacetate); mass spectroscopy: [M+H]⁺=442.

### Example 1.4

### 8-{2-[1,1-dimethyl-3-(3-methyl-2-oxo-2,3-dihydro-benzimidazol-1-yl)-propylamino]-1-hydroxy-ethyl}-6-hydroxy-4H-benzo[1,4]oxazin-3-one

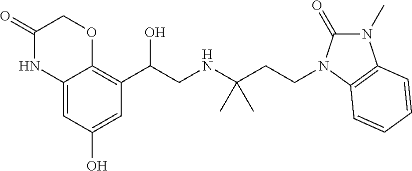

Prepared according to General Method 2 from 357 mg (1 mmol) 6-benzyloxy-8-(2-ethoxy-1,2-dihydroxy-ethyl)-4H-benzo[1,4]oxazin-3-one and 233 mg (1 mmol) 1-(3-amino-3-methyl-butyl)-3-methyl-1,3-dihydro-benzimidazol-2-one.

Yield: 270 mg (61%); mass spectroscopy: [M+H]⁺=441.

### Example 1.5

### 8-{2-[1,1-dimethyl-3-(2-oxo-2,3-dihydro-benzimidazol-1-yl)-propylamino]-1-hydroxy-ethyl}-6-hydroxy-4H-benzo[1,4]oxazin-3-one

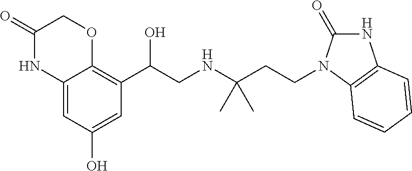

The target compound is obtained according to General Method 2 from 357 mg (1 mmol) 6-benzyloxy-8-(2-ethoxy-1,2-dihydroxy-ethyl)-4H-benzo[1,4]oxazin-3-one and 219 mg (1 mmol) 1-(3-amino-3-methyl-butyl)-1,3-dihydro-benzimidazol-2-one.

Yield: 187 mg (44%); mass spectroscopy: [M+H]⁺=427.

### Example 1.6

### 8-{2-[1,1-dimethyl-4-(2-oxo-2,3-dihydro-benzimidazol-1-yl)-butylamino]-1-hydroxy-ethyl}-6-hydroxy-4H-benzo[1,4]oxazin-3-one

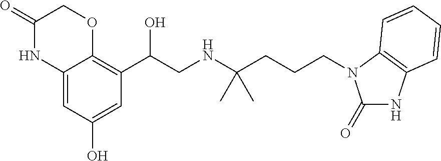

Prepared according to General Method 2 from 357 mg (1 mmol) 6-benzyloxy-8-(2-ethoxy-1,2-dihydroxy-ethyl)-4H-benzo[1,4]oxazin-3-one and 233 mg (1 mmol) 1-(4-amino-4-methyl-pentyl)-1,3-dihydro-benzimidazol-2-one.

Yield: 192 mg (44%); mass spectroscopy: [M+H]⁺=441.

### Example 1.7

### 8-{2-[1,1-dimethyl-3-(2-oxo-benzoxazol-3-yl)-propylamino]-1-hydroxy-ethyl}-6-hydroxy-4H-benzo[1,4]oxazin-3-one

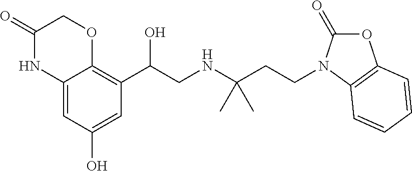

Prepared according to General Method 1 from 357 mg (1 mmol) 6-benzyloxy-8-(2-ethoxy-1,2-dihydroxy-ethyl)-4H-benzo[1,4]oxazin-3-one and 220 mg (1 mmol) 3-(3-amino-3-methyl-butyl)-3H-benzoxazol-2-one.

Yield: 227 mg (42%, trifluoroacetate); mass spectroscopy: [M+H]⁺=428.

### Example 1.8

### 7-{2-[1,1-dimethyl-3-(2-oxo-2,3-dihydro-benzimidazol-1-yl)-propylamino]-1-hydroxy-ethyl}-5-hydroxy-3H-benzoxazol-2-one

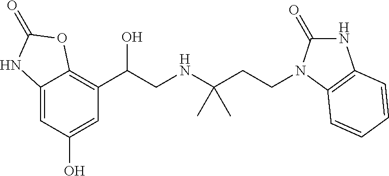

a) 5-benzyloxy-7-{2-[1,1-dimethyl-3-(2-oxo-2,3-dihydro-benzimidazol-1-yl)-propylamino]-1-hydroxy-ethyl}-3H-benzoxazol-2-one

343 mg (1 mmol) 5-benzyloxy-7-(2-ethoxy-2-hydroxy-acetyl)-3H-benzoxazol-2-one and 219 mg (1 mmol) 1-(3-amino-3-methyl-butyl)-1,3-dihydro-benzimidazol-2-one are stirred in 15 mL ethanol for 1.5 hours at 80° C. After cooling to ambient temperature, 80 mg (2 mmol) sodium borohydride are added and the mixture is stirred for 2 hours. The reaction mixture is acidified with 3 mL of 1 molar hydrochloric acid solution, stirred for 10 minutes and made alkaline with potassium carbonate solution. It is extracted with ethyl acetate, the organic phases are dried with sodium sulphate and the solvent is distilled off. The residue is purified by chromatography on a silica gel column (dichloromethane/methanol gradient). Beige solid. Yield: 340 mg (68%); mass spectroscopy [M+H]⁺=503.

b) 7-{2-[1,1-dimethyl-3-(2-oxo-2,3-dihydro-benzimidazol-1-yl)-propylamino]-1-hydroxy-ethyl}-5-hydroxy-3H-benzoxazol-2-one

320 mg (0.64 mmol) 5-benzyloxy-7-{2-[1,1-dimethyl-3-(2-oxo-2,3-dihydro-benzimidazol-1-yl)-propylamino]-1-hydroxy-ethyl}-3H-benzoxazol-2-one are dissolved in 12 ml of methanol and hydrogenated at ambient temperature with palladium on charcoal as catalyst. The catalyst is separated off and the filtrate is freed from the solvent. Beige solid. Yield: 150 mg (57%); mass spectroscopy [M−H]⁺=411.

### Example 1.9

### 8-{2-[3-(3-benzyl-2-oxo-2,3-dihydro-benzimidazol-1-yl)-1,1-dimethyl-propylamino]-1-hydroxy-ethyl}-6-hydroxy-2,2-dimethyl-4H-benzo[1,4]oxazin-3-one

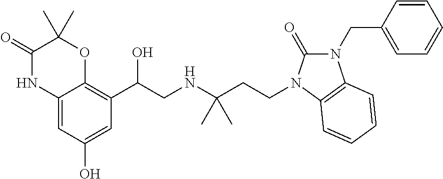

a) 1-(3-amino-3-methyl-butyl)-3-benzyl-1,3-dihydro-benzimidazol-2-one

Tert-butyl[1,1-dimethyl-3-(2-oxo-2,3-dihydro-benzimidazol-1-yl)-propyl]-carbamate, benzyl chloride and potassium-tert-butoxide are stirred overnight at ambient temperature in dimethylsulphoxide. The alkylation product tert-butyl[3-(3-benzyl-2-oxo-2,3-dihydro-benzimidazol-1-yl)-1,1-dimethyl-propyl]-carbamate obtained from the reaction is subsequently treated with trifluoroacetic acid/dichloromethane in order to cleave the protective group. Mass spectroscopy [M+H]⁺=310.

b) 8-{2-[3-(3-benzyl-2-oxo-2,3-dihydro-benzimidazol-1-yl)-1,1-dimethyl-propylamino]-1-hydroxy-ethyl}-6-hydroxy-2,2-dimethyl-4H-benzo[1,4]oxazin-3-one

385 mg (1 mmol) 6-benzyloxy-8-(2-ethoxy-2-hydroxy-acetyl)-2,2-dimethyl-4H-benzo[1,4]oxazin-3-one and 423 mg (1 mmol) 1-(3-amino-3-methyl-butyl)-3-benzyl-1,3-dihydro-benzimidazol-2-one are reacted and worked up according to General Method 1.

Yield: 39 mg (6%, trifluoroacetate); mass spectroscopy [M+H]⁺=545.

### Example 1.10

### 8-{2-[3-(3-cyclopropylmethyl-2-oxo-2,3-dihydro-benzimidazol-1-yl)-1,1-dimethyl-propylamino]-1-hydroxy-ethyl}-6-hydroxy-4H-benzo[1,4]oxazin-3-one

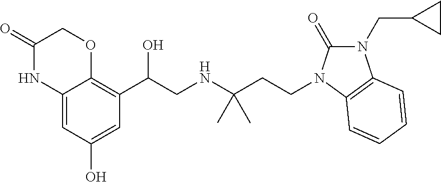

a) 1-(3-amino-3-methyl-butyl)-3-cyclopropylmethyl-1,3-dihydro-benzimidazol-2-one

The reaction of tert-butyl[1,1-dimethyl-3-(2-oxo-2,3-dihydro-benzimidazol-1-yl)-propyl]-carbamate with (chloromethyl)-cyclopropane and potassium-tert-butoxide in dimethylsulphoxide at ambient temperature yields tert-butyl[3-(3-cyclopropylmethyl-2-oxo-2,3-dihydro-benzimidazol-1-yl)-1,1-dimethyl-propyl]carbamate. Then the protective group of the alkylation product is cleaved by treating with trifluoroacetic acid in dichloromethane.

Mass spectroscopy [M+H]⁺=274.

b) 8-{2-[3-(3-cyclopropylmethyl-2-oxo-2,3-dihydro-benzimidazol-1-yl)-1,1-dimethyl-propylamino]-1-hydroxy-ethyl}-6-hydroxy-4H-benzo[1,4]oxazin-3-one

165 mg (0.5 mmol) 6-benzyloxy-8-(2-ethoxy-2-hydroxy-acetyl)-4H-benzo[1,4]oxazin-3-one and 194 mg (0.5 mmol) 1-(3-amino-3-methyl-butyl)-3-cyclopropylmethyl-1,3-dihydro-benzimidazol-2-one are dissolved in 8 mL ethanol and stirred for 1.5 hours at 80° C. The mixture is left to cool to ambient temperature, 19 mg (0.5 mmol) sodium borohydride are added and the mixture is stirred for another 2 hours. The reaction mixture is acidified with 1 molar hydrochloric acid, stirred for 10 minutes and made alkaline with potassium carbonate solution. Ethyl acetate is added and the aqueous phase is separated by filtration through kieselguhr. The organic phase is freed from the solvent and the residue is suspended in acetonitrile/water. The subsequent debenzylation is carried out analogously to General Method 1.

Yield: 77 mg (26%, trifluoroacetate); mass spectroscopy [M+H]⁺=481.

### Example 1.11

### 5-{2-[1,1-dimethyl-3-(2-oxo-2,3-dihydro-benzimidazol-1-yl)-propylamino]-1-hydroxy-ethyl}-7-hydroxy-1H-quinolin-2-one

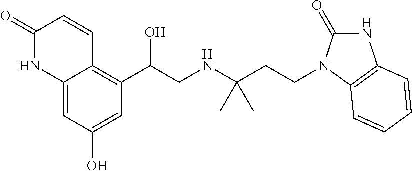

a) 7-benzyloxy-5-{2-[1,1-dimethyl-3-(2-oxo-2,3-dihydro-benzimidazol-1-yl)-propylamino]-1-hydroxy-ethyl}-1H-quinolin-2-one

121 mg (0.413 mmol) 7-benzyloxy-5-oxiranyl-1H-quinolin-2-one, 125 mg (0.570 mmol) 1-(3-amino-3-methyl-butyl)-1,3-dihydro-benzimidazol-2-one and 0.4 mL isopropanol are combined and irradiated with microwaves for 30 minutes at 135° C. The reaction mixture is combined with ethyl acetate and 0.5 molar tartaric acid, during which time a solid is precipitated. The solid and the aqueous phase are separated and water, dichloromethane and methanol are added. The aqueous phase is extracted with dichloromethane and the combined dichloromethane phases are dried and freed from the solvent. The residue is combined with hydrochloric acid in ethyl acetate, the solvent is distilled off and the residue is stirred in ethyl acetate. White solid.

Yield: 87 mg (38%, hydrochloride); mass spectroscopy: [M+H]⁺=513.

b) 5-{2-[1,1-dimethyl-3-(2-oxo-2,3-dihydro-benzimidazol-1-yl)-propylamino]-1-hydroxy-ethyl}-7-hydroxy-1H-quinolin-2-one

71 mg (0.129 mmol) 7-benzyloxy-5-[2-[1,1-dimethyl-3-(2-oxo-2,3-dihydro-benzimidazol-1-yl)-propylamino]-1-hydroxy-ethyl}-1H-quinolin-2-one hydrochloride are dissolved in methanol and hydrogenated at normal pressure with palladium on charcoal as catalyst. The catalyst is separated off by filtration through Celite and the filtrate is freed from the solvent. Stirring the residue with ethyl acetate yields the product in the form of a solid.

Yield: 31 mg (52%, hydrochloride); mass spectroscopy: [M+H]⁺=423.

### Example 1.12

### 6-hydroxy-8-{1-hydroxy-2-[4-(4-methoxy-2-oxo-2,3-dihydro-benzimidazol-1-yl)-1,1-dimethyl-butylamino]-ethyl}-4H-benzo[1,4]oxazin-3-one

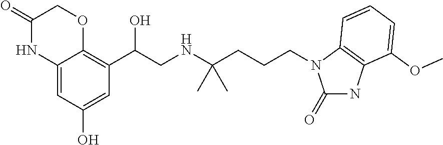

a) 6-benzyloxy-8-{1-hydroxy-2-[4-(4-methoxy-2-oxo-2,3-dihydro-benzimidazol-1-yl)-1,1-dimethyl-butylamino]-ethyl}-4H-benzo[1,41oxazin-3-one

200 mg (0.667 mmol) 1-(3-amino-3-methyl-butyl)-4-methoxy-1,3-dihydro-benzimidazol-2-one hydrochloride and 120 μL (0.733 mmol) triethylamine in 5 mL THF are stirred for 30 minutes and then combined with 200 mg (0.666 mmol) 6-benzyloxy-8-(2-ethoxy-2-hydroxy-acetyl)-4H-benzo[1,4]oxazin-3-one. After 2 hours the reaction mixture is cooled to 10° C. and 60 mg (2.76 mmol) lithium borohydride are added. The mixture is stirred for one hour at ambient temperature, then cooled to 10° C. and combined with 15 mL water. The organic phase is extracted with dichloromethane and the combined organic extracts are dried and freed from the solvent. The oil remaining is dissolved in ethyl acetate and adjusted to pH 2 with hydrochloric acid in ethyl acetate. The solvent is distilled off and the residue is stirred with dichloromethane/diethyl ether.

Yield: 130 mg (35%, hydrochloride); mass spectroscopy: [M+H]⁺=561.

b) 6-hydroxy-8-{1-hydroxy-2-[4-(4-methoxy-2-oxo-2,3-dihydro-benzimidazol-1-yl)-1,1-dimethyl-butylamino]-ethyl}-4H-benzo[1,41oxazin-3-one

130 mg (0.213 mmol) 6-benzyloxy-8-{1-hydroxy-2-[4-(4-methoxy-2-oxo-2,3-dihydro-benzimidazol-1-yl)-1,1-dimethyl-butylamino]-ethyl}-4H-benzo[1,4]oxazin-3-one hydrochloride are dissolved in methanol and hydrogenated with palladium on charcoal as catalyst at normal pressure. The catalyst is filtered off through Celite, the filtrate is freed from the solvent and the residue is stirred with ethyl acetate. Solid.

Yield: 50 mg (45%, hydrochloride); mass spectroscopy: [M+H]⁺=471.

### Example 1.13

### 6-hydroxy-8-{1-hydroxy-2-[4-(5-methoxy-3-methyl-2-oxo-2,3-dihydro-benzimidazol-1-yl)-1,1-dimethyl-butylamino]-ethyl}-4H-benzo[1,4]oxazin-3-one

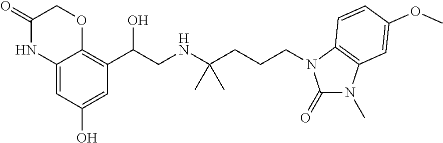

Prepared from 1-(3-amino-3-methyl-butyl)-5-methoxy-3-methyl-1,3-dihydro-benzimidazol-2-one and 6-benzyloxy-8-(2-ethoxy-2-hydroxy-acetyl)-4H-benzo[1,4]oxazin-3-one analogously to the method described for Example 1.12. Mass spectroscopy: [M+H]⁺=485.

### Example 1.14

### 8-{2-[4-(6-fluoro-3-methyl-2-oxo-2,3-dihydro-benzimidazol-1-yl)-1,1-dimethyl-butylamino]-1-hydroxy-ethyl}-6-hydroxy-4H-benzo[1,4]oxazin-3-one

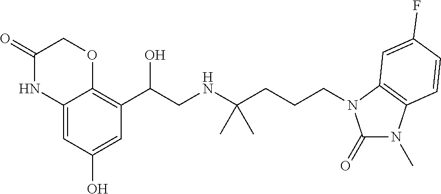

a) 6-benzyloxy-8-{2-[4-(6-fluoro-3-methyl-2-oxo-2,3-dihydro-benzimidazol-1-yl)-1,1-dimethyl-butylamino]-1-hydroxy-ethyl}-4H-benzo[1,4]oxazin-3-one

200 mg (0.754 mmol) 3-(4-amino-4-methyl-pentyl)-5-fluoro-1-methyl-1,3-dihydro-benzimidazol-2-one hydrochloride and 237 mg (0.663 mmol) 6-benzyloxy-8-(2-ethoxy-2-hydroxy-acetyl)-4H-benzo[1,4]oxazin-3-one are reacted analogously to the procedure laid down for Example 1.12a. The final purification is carried out by chromatography on a silica gel column. Yield: 164 mg (44%); mass spectroscopy: [M+H]⁺=563.

b) 8-{2-[4-(6-fluoro-3-methyl-2-oxo-2,3-dihydro-benzimidazol-1-yl)-1,1-dimethyl-butylamino]-1-hydroxy-ethyl}-6-hydroxy-4H-benzo[1,4]oxazin-3-one

164 mg (0.274 mmol) 6-benzyloxy-8-{2-[4-(6-fluoro-3-methyl-2-oxo-2,3-dihydro-benzimidazol-1-yl)-1,1-dimethyl-butylamino]-1-hydroxy-ethyl}-4H-benzo[1,4]oxazin-3-one are debenzylated analogously to the procedure laid down for Example 1.12b. For purification the crude product is stirred with ethyl acetate. Mass spectroscopy: [M+H]⁺=473.

### Example 1.15

### 8-{2-[4-(7-fluoro-3-methyl-2-oxo-2,3-dihydro-benzimidazol-1-yl)-1,1-dimethyl-butylamino]-1-hydroxy-ethyl}-6-hydroxy-4H-benzo[1,4]oxazin-3-one

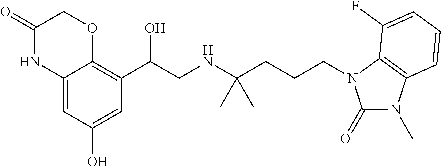

a) 6-benzyloxy-8-{2-[4-(7-fluoro-3-methyl-2-oxo-2,3-dihydro-benzimidazol-1-yl)-1,1-dimethyl-butylamino]-1-hydroxy-ethyl}-4H-benzo[1,4]oxazin-3-one

200 mg (0.663 mmol) 3-(4-amino-4-methyl-pentyl)-4-fluoro-1-methyl-1,3-dihydro-benzimidazol-2-one hydrochloride and 237 mg (0.663 mmol) 6-benzyloxy-8-(2-ethoxy-2-hydroxy-acetyl)-4H-benzo[1,4]oxazin-3-one are reacted analogously to the procedure laid down for preparing Example 1.12a. The final purification of the product is carried out by chromatography on a silica gel column.

Yield: 68 mg (17%); mass spectroscopy: [M+H]⁺=563.

b) 8-{2-[4-(7-fluoro-3-methyl-2-oxo-2,3-dihydro-benzimidazol-1-yl)-1,1-dimethyl-butylamino]-1-hydroxy-ethyl}-6-hydroxy-4H-benzo[1,4]oxazin-3-one

68 mg (0.121 mmol) 6-benzyloxy-8-{2-[4-(7-fluoro-3-methyl-2-oxo-2,3-dihydro-benzimidazol-1-yl)-1,1-dimethyl-butylamino]-1-hydroxy-ethyl}-4H-benzo[1,4]oxazin-3-one are debenzylated using the method described for Example 1.12b. For purification the crude product is stirred in ethyl acetate. Yield: 60 mg; mass spectroscopy: [M+H]⁺=474.

We claim:

1. A pharmaceutical composition comprising one or more compounds of formula 1 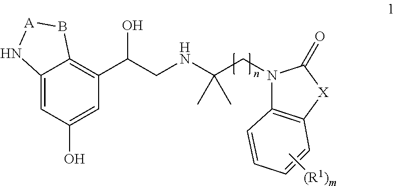
wherein
n denotes 1, 2, 3 or 4;
m denotes 1, 2 or 3;
X denotes CH₂, CO, NR², S or O;
A denotes a double-bonded group chosen from CO, SO and SO₂;
B denotes a double-bonded group chosen from O, S, CH₂, CR³R⁴—O, CR³R⁴—S, NR⁵, CR³R⁴—NR⁵, CH═CH or CH₂—CH₂;
R¹ denotes H, C₁₋₆-alkyl, C₂₋₆-alkenyl, C₂₋₆-alkynyl, C₃₋₆-cycloalkyl, C₁₋₆-haloalkyl, O-C₁₋₆-haloalkyl, halogen, OH, CN, NO₂, O—C₁₋₆-alkyl, COON or COO—C₁₋₄-alkyl;
R² denotes H, C₁₋₆-alkyl, C₁₋₄-alkylene-C₆-C₁₀-aryl or C₁₋₄-alkylene-C₃₋₆-cycloalkyl;
R³ denotes H or C₁₋₆-alkyl;
R⁴ denotes H or C₁₋₆-alkyl;
R⁵ denotes H or C₁₋₆-alkyl; and
at least one other active substance 2.

2. The pharmaceutical composition according to claim 1, wherein the additional active substance 2 is one or more compounds selected from the group consisting of anticholinergics (2a), PDEIV-inhibitors (2b), steroids (2c), LTD4-antagonists (2d) and EGFR inhibitors (2e) as a further active substance 2.

3. The pharmaceutical composition according to claim 1 comprising one or more compounds of formula 1, wherein
n denotes 1, 2 or 3;
m denotes 1, 2 or 3;
X denotes CH₂, CO, NR², S or O;
A denotes CO;
B denotes a double-bonded group chosen from O, S, CH₂, CR³R⁴—O, CR³R⁴—S, NR⁵, CR³R⁴—NR⁵, CH═CH and CH₂—CH₂;
R¹ denotes H, C₁₋₄-haloalkyl, cyclopropyl, cyclohexyl, halogen, OH, O—C₁₋₄-alkyl, COON or COOMe;
R² denotes H, C₃₋₆-cycloalkyl-methyl;
R³ denotes H or C₁₋₄-alkyl;
R⁴ denotes H or C₁₋₄-alkyl; and
R⁵ denotes H or C₁₋₄-alkyl.

4. The pharmaceutical composition according to claim 1 comprising one or more compounds of formula 1, wherein
n denotes 2 or 3;
m denotes 1, 2 or 3;
X denotes CH₂, CO, NR², S or O;
A denotes CO;
B denotes a double-bonded group chosen from CH₂—O, CH═CH or CH₂—CH₂;
R¹ denotes H, methyl, ethyl, propyl, CF₃, CH₂F, CH₂CF₃, fluorine, chlorine, bromine, OH, methoxy, ethoxy, COON or COOMe;
R² denotes H, methyl, ethyl or propyl.

5. The pharmaceutical composition according to claim 1, wherein the one or more compounds of formula 1 are in the form of the individual optical isomers, mixtures of the individual enantiomers or racemates.

6. The pharmaceutical composition according to claim 1, wherein the one or more compounds of formula 1 are in the form of the acid addition salts with pharmacologically acceptable acids as well as optionally in the form of the solvates and/or hydrates.

7. The pharmaceutical composition according to claim 1, wherein the additional active substance 2 is an anticholinergic (2a).

8. The pharmaceutical composition according to claim 1, wherein the additional active substance 2 is a PDE IV-inhibitor (2b).

9. The pharmaceutical composition according to claim 1, wherein the additional active substance 2 is a steroid (2c).

10. The pharmaceutical composition according to claim 1, wherein the additional active substance 2 is an LTD4-antagonist (2d).

11. The pharmaceutical composition according to claim 1, wherein the additional active substance 2 is an EGFR inhibitor (2e).

12. The pharmaceutical composition according to claim 1, comprising therapeutically effective amounts of one or more of compounds formula 1, therapeutically effective amounts of an anticholinergic (2a), therapeutically effective amounts of a PDEIV inhibitor (2b), and optionally a pharmaceutically acceptable carrier.

13. The pharmaceutical composition according to claim 1, wherein the additional active substance 2 comprises an anticholinergic (2a), a steroid (2c), and optionally a pharmaceutically acceptable carrier.

14. The pharmaceutical composition according to claim 1, comprising therapeutically effective amounts of one or more of compounds formula 1, therapeutically effective amounts of an anticholinergic (2a), therapeutically effective amounts of an LTD4-antagonist (2d), and optionally a pharmaceutically acceptable carrier.

15. The pharmaceutical composition according to claim 1, comprising therapeutically effective amounts of one or more of compounds formula 1, therapeutically effective amounts of an anticholinergic (2a), therapeutically effective amounts of an EGFR inhibitor (2e), and optionally a pharmaceutically acceptable carrier.

16. The pharmaceutical composition according to claim 1, comprising therapeutically effective amounts of one or more of compounds formula 1, therapeutically effective amounts of a PDEIV inhibitor (2b), therapeutically effective amounts of a steroid (2c), and optionally a pharmaceutically acceptable carrier.

17. The pharmaceutical composition according to claim 1, comprising therapeutically effective amounts of one or more of compounds formula 1, therapeutically effective amounts of a PDEIV inhibitor (2b), therapeutically effective amounts of an LTD4-antagonist (2d), and optionally a pharmaceutically acceptable carrier.

18. The pharmaceutical composition according to claim 1, comprising therapeutically effective amounts of one or more of compounds formula 1, therapeutically effective amounts of a PDEIV inhibitor (2b), therapeutically effective amounts of an EGFR inhibitor (2e), and optionally a pharmaceutically acceptable carrier.

19. The pharmaceutical composition according to claim 1, comprising therapeutically effective amounts of one or more of compounds formula 1, therapeutically effective amounts of a steroid (2c), therapeutically effective amounts of an LTD4-antagonist (2d), and optionally a pharmaceutically acceptable carrier.

20. The pharmaceutical composition according to claim 1, comprising therapeutically effective amounts of one or more of compounds formula 1, therapeutically effective amounts of a steroid (2c), therapeutically effective amounts of an EGFR inhibitor (2e), and optionally a pharmaceutically acceptable carrier.

21. The pharmaceutical composition according to claim 1, comprising therapeutically effective amounts of one or more of compounds formula 1, therapeutically effective amounts of an LTD4-antagonist (2d), therapeutically effective amounts of an EGFR inhibitor (2e), and optionally a pharmaceutically acceptable carrier.

22. The pharmaceutical composition according to claim 1, further comprising a pharmaceutically acceptable carrier.

23. The pharmaceutical composition according to claim 1, wherein the composition does not comprise any pharmaceutically acceptable carrier.

24. The pharmaceutical composition according to claim 1, wherein the composition is in the form of a formulation suitable for inhalation.

25. The pharmaceutical composition according to claim 24, wherein the inhalation formulation is selected from the group consisting of inhalable powders, propellant-driven metered-dose aerosols and propellant-free inhalable solutions or suspensions.

26. The pharmaceutical composition according to claim 25, wherein the inhalable powder comprises one or more compounds of formula 1 and active substance 2 in admixture with suitable physiologically acceptable excipients selected from the group consisting of monosaccharides, disaccharides, oligo- and polysaccharides, polyalcohols, salts, and mixtures thereof.

27. The pharmaceutical composition according to claim 25, wherein the propellant-driven inhalable aerosol comprises one or more compounds of formula 1 and active substance 2 in dissolved or dispersed form.

28. The pharmaceutical composition according to claim 27, wherein the inhalable aerosol comprises as the propellant gas hydrocarbons selected from n-propane, n-butane or isobutene, or halohydrocarbons selected from chlorinated and/or fluorinated derivatives of methane, ethane, propane, butane, cyclopropane or cyclobutane.

29. The pharmaceutical composition according to claim 28, wherein the propellant gas is selected from TG11, TG12, TG134a, TG227 or mixtures thereof.

30. The pharmaceutical composition according to claim 25, wherein the propellant-free inhalable solution or suspension comprises as a solvent water, ethanol or a mixture thereof.

31. A method of treating inflammatory and obstructive respiratory complaints, circulatory shock (vasodilatation and increasing the heart volume), skin irritations and inflammation, inhibiting premature labour in midwifery (tocolysis), restoring sinus rhythm in the heart in atrioventricular block, and correcting bradycardic heart rhythm disorders (antiarrhythmic) comprising administering to a patient in need thereof a therapeutically effect amount of a composition according to claim 1.

32. The method according to claim 31, wherein the respiratory complaint is selected from the group consisting of obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

33. The method according to claim 32, wherein the obstructive pulmonary diseases are selected from among bronchial asthma, paediatric asthma, severe asthma, acute asthma attacks, chronic bronchitis and COPD (chronic obstructive pulmonary disease).

34. The method according to claim 32, wherein the pulmonary emphysema which has its origins in COPD or α1-proteinase inhibitor deficiency.

35. The method according to claim 32, wherein the restrictive pulmonary disease is selected from allergic alveolitis, restrictive pulmonary diseases triggered by work-related noxious substances, such as asbestosis or silicosis, and restriction caused by lung tumours selected from lymphangiosis carcinomatosa, bronchoalveolar carcinoma and lymphomas.

36. The method according to claim 32, wherein the interstitial pulmonary diseases are selected from pneumonia caused by infections selected from viruses, bacteria, fungi, protozoa, helminths or other pathogens, pneumonitis caused by various factors selected from aspiration or left heart insufficiency, radiation-induced pneumonitis or fibrosis, collagenoses selected from lupus erythematodes, systemic sclerodermy or sarcoidosis, or granulomatoses selected from Boeck's disease, idiopathic interstitial pneumonia or idiopathic pulmonary fibrosis (IPF).

37. The method according to claim 32, wherein the respiratory complaint is cystic fibrosis or mucoviscidosis.

38. The method according to claim 32, wherein the respiratory complaint is bronchitis caused by bacterial or viral infection, allergic bronchitis or toxic bronchitis.

39. The method according to claim 32, wherein the respiratory complaint is bronchiectasis.

40. The method according to claim 32, wherein the respiratory complaint is ARDS (adult respiratory distress syndrome).

41. The method according to claim 32, wherein the respiratory complaint is pulmonary oedema.
